(19) United States
(12) Patent Application Publication
(10) Pub. No.: US 2024/0390432 A1
(43) Pub. Date: Nov. 28, 2024

(54) ENGINEERED VECTORS AND ORGANISMS CONTAINING THE SAME FOR ISOFLAVONE CONVERSION IN THE GUT

(71) Applicant: Board of Regents, The University of Texas System, Austin, TX (US)

(72) Inventor: Justyn JAWORSKI, Arlington, TX (US)

(21) Appl. No.: 18/697,325

(22) PCT Filed: Sep. 30, 2022
(86) PCT No.: PCT/US2022/077338

§ 371 Date: Mar. 29, 2024

Related U.S. Application Data
(60) Provisional application No. 63/250,717, filed on Sep. 30, 2021.

(51) Int. Cl. A61K 35/741; A61P 9/00; C12N 1/20; C12N 9/02; C12N 15/70; C12R 1/19

(52) U.S. Cl. CPC A61K 35/741; A61P 9/00; C12N 1/20; C12N 9/0071; C12N 15/70; C12R 2001/19; C12Y 114/14

(57) ABSTRACT

Generally, the present disclosure contemplates engineered vectors, organisms (e.g., bacteria or bacteriophage) containing the same, and methods for treating the gut of mammalian species by providing the organism containing the engineered vector to the gut of a mammal. Example engineered organisms can include one or more genes isolated from microbial populations for promoting isoflavone metabolism.

### CROSS REFERENCE TO RELATED APPLICATIONS

The present application claims priority to U.S. Provisional Patent Application No. 63/250,717, filed Sep. 30, 2021, the entire contents and substance of which are incorporated herein by reference.

### INCORPORATION BY REFERENCE OF SEQUENCE LISTING OR TABLE

The material in the accompanying sequence listing is hereby incorporated by reference in its entirety into this application. The accompanying file, named 060060-00204.xml, was created on Sep. 21, 2022 and is 35 kB.

### FIELD

The present disclosure is directed to engineered transmissible gene constructs (e.g., vectors). More particularly, the disclosure provides vectors encoding isoflavone metabolism pathways that can be included in bacteria to produce probiotics for modifying mammalian gut phenotype.

### BACKGROUND

In the mammalian gut, dynamics of the environment, including continuous secretion of mucin, limit direct colonization of the underlying epithelium but permit high density microbial communities to reside. The diversity of species within and across hosts is believed to be spatially structured, and it is known that the high bacterial densities presented in the mammalian gut, up to 10³ CFU/ml for the stomach and duodenum, 10⁸ CFU/ml for the jejunum and ileum, and 10¹² CFU/g for the colon, may present opportunities for gene transfer. Gene transfer can occur between bacteria including different strains of bacteria, and the dynamics of gene transfer may be of particular importance for genes related to the ability to break down food and drugs, the emergence of antibiotic resistance, and communication with the host immune system.

Approaches to study gene transfer in the gut have included culture-based techniques and high-throughput fluorescent systems in which lab animals are fed bacteria containing genes for antibiotic resistance or fluorescent reporter genes to isolate transconjugants by selection or screening, respectively. A recent study used fluorescence-activated cell sorting (FACS) of the bacterial recipients of plasmids in a microbial community extracted from soil showing that this heterogeneous community provides a hot-spot for gene acquisition from phylogenetically distant groups as introduced plasmids were found to be hosted by very diverse bacteria. Data has supported that host range of plasmid recipients is donor-dependent, and it has also been shown that plasmid fitness effects (or costs on the host) can differ between bacteria ultimately impacting plasmid persistence in bacterial communities. Still needed though is an improvement of treatment modalities such as dosing schedule, dosing amount, and pre-treatment with antibiotics to observe the effect on plasmid persistence and uptake.

A natural isoflavone known as Equol is one such case of a microbially synthesized metabolite formed from dietary Daidzein. Equol has been found to be a selective agonist for estrogen receptor beta signalling indicated in reducing large artery stiffness. Unfortunately, only a fraction of the population possesses the intestinal bacteria capable of converting Daidzein from dietary soy into Equol. Such individuals with an Equol producing phenotype experience a range of health benefits with soy intake, including decreased risk of cardiovascular disease.

### SUMMARY

To address human health in view of the above, we have investigated, and herein disclose, means to engineer the intestinal microbiota toward an Equol producing phenotype by introduction of vectors (e.g., plasmids) to the gut bacteria for expression of the proteins daidzein reductase (DZNR), dihydrodaidzein racemase (DDRC), dihydrodaidzein reductase (DHDR), and tetrahydrodaidzein reductase (THDR). Here we disclose, in some embodiments, an engineered bacterial metabolic pathway for enzymatic conversion of the isoflavones Daidzein and Genistein to the products Equol and 5-Hydroxy-Equol respectively, and moreover we offer a strategy for controllable persistence of this engineered genetic construct among the gut bacteria.

In general, the present disclosure contemplates engineered vectors, organisms (e.g., bacteria or bacteriophage) containing the same, and methods for treating the gut of mammalian species by providing the organism containing the engineered vector to the gut of a mammal.

In one aspect, vectors including one or more genes each encoding a different protein are described herein. The one or more genes can have a nucleotide sequence according to SEQ ID NO. 1 which encodes a protein for Daidzein reductase (DZNR); according to SEQ ID NO. 2 which encodes a protein for Dihydrodaidzein racemase (DDRC); according to SEQ ID No. 3 which encodes a protein for Dihydrodaidzein reductase (DHDR); or according to SEQ ID NO. 4 which encodes a protein for Tetrahydrodaidzein reductase (THDR).

In another aspect, a bacterium of the genus Escherichia is disclosed herein, the bacterium including one or more vectors in accordance with the present disclosure. In some implementations the one or more vectors may include the genes encoding the proteins DZNR, DDRC, DHDR, THDR, or a combination thereof (e.g., DZNR and DDRC).

In still another aspect, a probiotic for the treatment of cardiovascular disease (CVD) is disclosed herein. The probiotic, in some embodiments, includes one or more engineered vectors having one or more genes encoding a protein for isoflavone metabolism.

In a further aspect, a method for modifying the phenotype of a mammalian gut is disclosed herein. The method, in some cases, includes introducing to the mammalian gut a probiotic that incorporates a bacterium including one or more vectors having one or more genes for isoflavone metabolism. In some implementations, the method can include a dosing regimen and/or adding a transduction agent (e.g., bacteriophage).

### BRIEF DESCRIPTION OF THE DRAWINGS

FIG. 1 schematically illustrates a model of the mammalian gut on exposure to plasmid donor bacteria in accordance with example embodiments of the present disclosure.

FIG. 2A illustrates a graph depicting carrier bacteria versus days after ending donor input in accordance with example aspects of the present disclosure.

FIG. 2B illustrates a graph depicting carrier bacteria versus days after ending donor input in accordance with example aspects of the present disclosure.

FIG. 3 illustrates a graph depicting carrier bacteria versus days after ending donor input in accordance with example aspects of the present disclosure.

FIG. 4 illustrates one example metabolic pathway for converting Daidzein to Equol according to example implementations of the present disclosure.

FIG. 5 illustrates two example plasmid constructs: P1 (left) and P2 (right) according to example implementations of the present disclosure.

FIG. 6 depicts a thin layer chromatograph of Daidzein metabolism upon exposure to expression products of P1 and P2.

FIG. 7 illustrates a cartoon depicting transfer of genes and subsequent infection of gut bacteria according to example implementations of the present disclosure.

FIG. 8 illustrates a cartoon depicting a two bacterium system for converting Daidzein to Equol according to an example implementation of the present disclosure.

FIG. 9A illustrates a photograph of a TLC plate run on material exposed to P1 plasmid products according to an example implementation of the present disclosure.

FIG. 9B illustrates a photograph of a TLC plate run on material exposed to P2 plasmid products according to an example implementation of the present disclosure.

FIG. 10A illustrates a graph depicting dihydrodaidzein formed versus time according to an example implementation of the present disclosure.

FIG. 10B illustrates a graph depicting Equol formed versus time according to an example implementation of the present disclosure.

FIG. 11 illustrates a graph depicting formation of Equol in mixed populations of Nissle P1 and Nissle P2 strains when provided 9 μM Daidzein substrate according to an example implementation of the present disclosure.

FIG. 12 illustrates a graph depicting Equol concentration versus the diet given to mice in accordance with example aspects of the present disclosure.

### DETAILED DESCRIPTION

Embodiments described herein can be understood more readily by reference to the following detailed description and examples. Elements, apparatus and methods described herein, however, are not limited to the specific embodiments presented in the detailed description and examples. It should be recognized that these embodiments are merely illustrative of the principles of the present disclosure. Numerous modifications and adaptations will be readily apparent to those of skill in the art without departing from the spirit and scope of the disclosure.

In addition, all ranges disclosed herein are to be understood to encompass any and all subranges subsumed therein. For example, a stated range of “1.0 to 10.0” should be considered to include any and all subranges beginning with a minimum value of 1.0 or more and ending with a maximum value of 10.0 or less, e.g., 1.0 to 5.3, 1 to 4, 3 to 7, 4.7 to 10.0, 3.6 to 7.9, or 5 to 8.

All ranges disclosed herein are also to be considered to include the end points of the range, unless expressly stated otherwise. For example, a range of “between 5 and 10,” “from 5 to 10,” or “5-10” should generally be considered to include the end points 5 and 10.

It is also to be understood that the article “a” or “an” refers to “at least one,” unless the context of a particular use requires otherwise.

In general, the present disclosure is directed to man-made vectors which include nucleic acid sequences encoding one or more proteins, carriers including one or more of the vectors described herein, methods for producing said vectors, and methods for improving gut health in a mammal which include introducing said vector to the mammal.

One example aspect of the present disclosure includes vectors which include one or more genes, the one or more genes having a nucleotide sequence encoding a protein selected from: Daidzein reductase (DZNR); Dihydrodaidzein racemase (DDRC); Dihydrodaidzein reductase (DHDR); or Tetrahydrodaidzein reductase (THDR).

As used herein, a vector is a transmissible genetic construct (e.g., a plasmid).

According to certain aspects of the present disclosure, a vector can be carried by a bacteria or bacteriophage to encapsulate and/or deliver the vector or vectors.

In some implementations, the nucleotide sequence encoding the protein can be derived from an organism such as L. garvieae, Coriobacteriaceae, or S. isoflavoniconvertens. For example, SEQ ID NOs 1-4 are nucleotide sequences derived from Lactococcus garvieae. However, for implementations of the present disclosure, genes encoding proteins for DZNR, DDRC, DHDR, or THDR may be derived from one or multiple organisms other than or in addition to Lactococcus garvieae.

For certain implementations, the nucleotide sequence derived from the organism can be modified (e.g., using codon optimization) to facilitate expression of the protein in a heterologous organism. For instance, codon optimization can be performed using one of the sequence ID listings herein (e.g., SEQ ID NO. 1) to generate an optimized SEQ ID NO. 1 for expression by Escherichia bacteria species.

In one example implementation, a vector can include two genes from Lactococcus garvieae. For instance, a vector can include the two genes from Lactococcus garvieae which encode the proteins DZNR and DHDR.

In some implementations, the vector can further include an antibiotic resistance gene. Alternatively, in certain implementations that vector can include no antibiotic resistance gene. One example advantage of a vector that does not include an antibiotic resistance gene is that this vector will not intentionally transmit antibiotic resistance to potentially pathogenic bacteria within the gut of mammalian populations.

Several non-limiting examples of antibiotic resistances genes can include a gene for resistance to ampicillin, kanamycin, streptomycin, penicillin, neomycin, gentamicin, chloramphenicol, erythromycin, zeocin, bleomycin, or tetracycline.

In some implementations, the vector can include a bacteriophage genome. An example vector in accordance with the present disclosure can include the nucleotide sequence encoding Daidzein reductase (DZNR) having the sequence of SEQ ID 1 or a codon optimized sequence thereof.

Another example vector in accordance with the present disclosure can include the nucleotide sequence encoding Dihydrodaidzein racemase (DDRC) having the sequence of SEQ ID 2 or a codon optimized sequence thereof.

A further example vector in accordance with the present disclosure can include the nucleotide sequence encoding Dihydrodaidzein reductase (DHDR) having the sequence of SEQ ID 3 or a codon optimized version thereof.

A still further example vector in accordance with the present disclosure can include the nucleotide sequence encoding Tetrahydrodaidzein reductase (THDR) having the sequence of SEQ ID 4 or a codon optimized version thereof.

One example implementation of the present disclosure can include a bacterium comprising one or more vectors according to the present disclosure, wherein one of the one or more vectors comprises the nucleotide sequences encoding DZNR and DDRC.

In certain example implementations, the bacterium including the one or more vectors can have a first plasmid and a second plasmid. According to an example aspect of the disclosure, the second plasmid can have a different nucleotide sequence from the first plasmid. For instance, the first plasmid may include the nucleotide sequences encoding DDRC and DZNR, and the second plasmid may include the nucleotide sequences encoding DHDR and THDR.

Additionally or alternatively, in some implementations a two bacterium system can be used where each bacterium includes a different plasmid (e.g., a first bacterium having the first plasmid and a second bacterium including the second plasmid).

In one example implementations, the one or more vectors can consist of one plasmid, and the one plasmid further includes the nucleotide sequences encoding DHDR, and THDR. Thus, the disclosure contemplates a bacterium having one plasmid that includes the nucleotide sequences encoding DZNR, DDRC, DDHDR, and THDR.

Several non-limiting examples of bacteria that can be used to contain the vector(s) described herein can include bacteria of Escherichia genus, Bacteroides genus, Bifidobacterium genus, Streptococcus genus, Bacillus, Enterococcus, or Lactobacillus genus.

For example, in some implementations, the bacteria of the Escherichia genus can be E. coli, in some implementations the bacteria of the Bacteroides genus can be B. intestinalis, the bacteria of the Bifidobacterium genus can in some implementations be B. longum, the bacteria of the Streptococcus genus can be S. thermophilus, the bacteria of the Bacillus genus can be B. subtilis, the bacteria of the Enterococcus genus can be E. faecium, and/or the bacteria of the Lactobacillus genus can be L. plantarum.

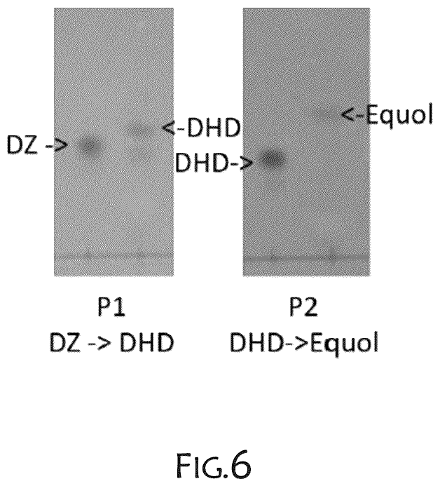

Another example implementation of the disclosure can include a bacterium from the genus Escherichia containing therein at least two stable plasmids for isoflavone metabolism, wherein said plasmids provide components (e.g., separate or unique components) for converting Daidzein to Equol, Genistein to 5-Hydroxy-Equol, or both. Isoflavone metabolism can be exemplified using several reaction pathways. For instance, FIG. 6 illustrates the multi-reaction mechanism from Daidzein to Equol, which uses three proteins to achieve transformation. According to example aspects of the present disclosure, genes encoding the three proteins can be included on at least two stable plasmids such as genes encoding DZNR and DDRC on a first stable plasmid and genes encoding DHDR and THDR on a second stable plasmid.

To further illustrate, in some embodiments, the separate isoflavone metabolism pathways that can be included in bacteria are provided in one or more genes described herein, such as the genes encoding a protein selected from: Daidzein reductase (DZNR), Dihydrodaidzein racemase (DDRC), Dihydrodaidzein reductase (DHDR), or Tetrahydrodaidzein reductase (THDR). In certain implementations, the genes can include a codon optimized nucleic acid sequence. In some such cases, the codon optimized nucleic acid sequence is generated to produce the same amino acid sequence. Any available techniques (e.g., online tool kits) may be used to generate the codon optimized nucleic acid sequence. For example, in some embodiments, the Integrated DNA Technologies (IDT) Codon Optimization Tool can be applied to design an optimal DNA sequence for expression of DZNR, DDRC, DHDR, and/or THDR in a host organism based on its codon usage.

In one example implementation, one of the at least two stable plasmids for metabolizing isoflavone includes the genes encoding DDRC and DZNR, wherein a second of the at least two stable plasmids for metabolizing isoflavone includes the genes encoding DHDR and THDR.

Another embodiment of the present disclosure can include a probiotic for the treatment of cardiovascular disease (CVD), the example probiotic including a bacterium according to the disclosure.

One example aspect of the probiotic is a dosage form, such as a dosage form that can be taken orally. Several example dosage forms of a probiotic described herein can include a liquid, a foodstuff, a capsule, or other acceptable form for ingestion.

A further example embodiment of the present disclosure can include a method for modifying the phenotype of a mammalian gut. The example method can comprise introducing to the mammalian gut a probiotic according to the present disclosure.

In some implementations, the method of modifying the phenotype of the mammalian gut can also include co-administering a transduction agent which can affect the transmission of vectors and/or uptake of vectors described herein.

Several non-limiting examples of transduction agents can include an antibiotic, a polycationic polymer (e.g., lipofectamine), a bacteriophage, a polycationic liposome, a polycationic nanoparticle, etc.

In some implementations, a method for modifying the phenotype of the mammalian gut described herein can be implemented so that introducing the probiotic to the mammalian gut is performed according to a dosage regimen.

For instance, in some methods the dosage regimen can include introducing the probiotic having a concentration of the bacterium to the mammalian gut at a dosing schedule over the course of a time period.

In an example implementation, the concentration of the bacterium is between 1×10⁶ to 1×10¹⁰ CFU/mL.

According to an aspect of the disclosure, the time period can be selected from: every 4 hours, every 6 hours, every 12 hours, every 24 hours, or every 48 hours.

Additionally or alternatively, the time period can be 1 day, 1 week, 1 month, or one year.

In one example embodiment, the probiotic provided to the mammalian gut can include: a first bacterium having a first plasmid comprising the nucleotide sequence or sequences encoding DZNR, DDRC, or both; and a second bacterium having a second plasmid comprising the nucleotide sequence or sequences encoding DHDR, THDR, or both. According to some example aspects of the disclosure, a ratio of the first bacterium to the second bacterium is no less than 9:1 and no greater than 1:9 such as 9:1 to 1:1, 1:1 to 1:9, or 1:1 to 1:5.

Another embodiment of the disclosure can include a method of producing a vector described herein. An example method can include: obtaining a cloning vector (e.g., a bacteriophage genome, phagemid, or plasmid); and inserting into the cloning vector the nucleotide sequence for Daidzein reductase (DZNR), Dihydrodaidzein racemase (DDRC), Dihydrodaidzein reductase (DHDR), or Tetrahydrodaidzein reductase (THDR). Moreover, in some such instances, the cloning vector does not include an antibacterial resistance gene.

According to certain aspects of the present disclosure, the cloning vector used to produce the man-made vector can include one or more restriction sites.

Further, inserting into the cloning vector the nucleotide sequence may include introducing a restriction enzyme to the cloning vector; providing a cloning sequence comprising the nucleotide sequence for Daidzein reductase (DZNR); Dihydrodaidzein racemase (DDRC); Dihydrodaidzein reductase (DHDR); or Tetrahydrodaidzein reductase (THDR); and ligating the cloning sequence to the cloning vector.

Another example aspect of producing the vector can include isolating the vector containing the nucleotide sequence.

In one aspect, the present invention will be better understood with reference to the following non-limiting examples and the drawings.

### EXAMPLES

The present examples provide aspects of embodiments of the present disclosure. These examples are not meant to limit embodiments solely to such examples herein, but rather to illustrate some possible implementations.

### Example 1

### Plasmid Dosage and Persistence in Murine Gut

### SUMMARY

Here we describe dosing strategies for introducing a plasmid to the bacterial community of the murine gut as evaluated on the ability of plasmid carriers to persist within the mouse gastrointestinal tract. Mice were provided carrier bacteria (E. coli) possessing a plasmid harboring a reporter gene and antibiotic resistance gene. Variations in dosage strategies included amount of donor bacteria, pretreatment of the gut with broad-spectrum antibiotics, and the extent of time in which the mice were provided the donor bacteria. Bacteria were collected from fecal pellets for analysis by plate assay to determine the proportion of bacteria carrying the introduced plasmid. As described further below, longer dosing regimens with plasmid donor bacteria can lead to a significantly increased duration for persistence of the plasmid within the gut. The amount of donor bacteria and use of antibiotic pretreatment marginally influenced the duration of plasmid persistence but did influence short-term abundance. Herein we describe a temporal assessment of plasmid introduced to the murine gut.

Mice were provided bacteria containing a genetic construct, known as a plasmid, and were examined for the persistence of the plasmid in the gut. Long-term persistence of the plasmid within the gut was identified when using extended schedules of dosing with donor bacteria carrying the plasmid. The use of higher amounts of dosing influenced short term abundance of the plasmid carrying bacteria in the gut.

### INTRODUCTION

In the mammalian gut, dynamics of the environment, including continuous secretion of mucin, limit direct colonization of the underlying epithelium but permit high density microbial communities to reside. The diversity of species within and across hosts is believed to be spatially structured, as it is well known that the high bacterial densities presented in the mammalian gut, up to 10³ CFU/ml for the stomach and duodenum, 10⁸ CFU/ml for the jejunum and ileum, and 10¹² CFU/g for the colon, are conducive to gene transfer. Gene transfer can occur among bacteria, and the importance of understanding these dynamics becomes clear when considering the possible impact such gene transfer events can have on the ability to break down food and drugs, the emergence of antibiotic resistance, and the communication with the host immune system. To explore the possibility of introducing new genetic elements to existing gut bacteria for modifying their metabolic capabilities, it is important to understand the transmission and persistence of mobile genetic constructs within the gut.

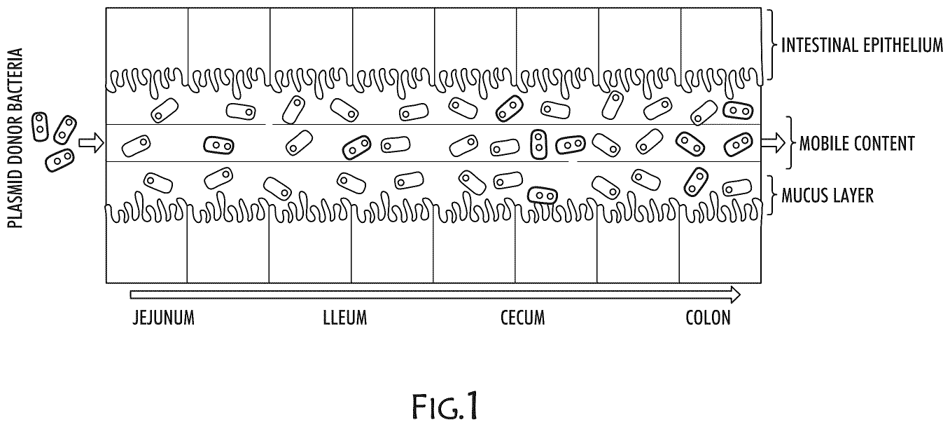

In this Example, we describe the introduction of plasmid to the murine gut to evaluate its availability to persist within the microbial community of the gastrointestinal tract in relation to dosage duration of donor bacteria and usage of antibiotic pre-treatment. See, e.g., FIG. 1. Approaches to study gene transfer in the gut have included culture-based techniques and high-throughput fluorescent systems in which lab animals are fed bacteria containing genes for antibiotic resistance or fluorescent reporter genes to isolate transconjugants by selection or screening, respectively. Data has supported that host range of plasmid recipients is donor dependent and it has also been shown that plasmid fitness effects (or costs on the host) can differ between bacteria ultimately impacting plasmid persistence in bacterial communities. Herein we describe the role of dosing schedule, dosing amount, and pre-treatment with antibiotic to reduce competing bacterial communities before introduction of a donor bacteria to observe the effect on plasmid persistence and uptake. In addition, we describe whether using extended timeframes for continuous dosing increases the chance of plasmid transfer from the introduced donor to the residing bacteria of the gut.

In this Example, we specifically describe the persistence of a plasmid carrying a set of antibiotic selectable and fluorescently screen-able genetic markers introduced to the murine gut within E. coli as the donor. To test the hypothesis that the persistence of the plasmid would be dependent upon the residence time of the donor and the existing recipient population, plate-based culture assays of fecal samples were carried out to quantify the amount of plasmid carrier bacteria with respect to variations in antibiotic pretreatment duration and dosage schedule as well as amount for the E. coli donor. As described, we have identified that the persistence of an introduced plasmid can be extended significantly by regulating the dosage schedule, and antibiotic pre-treatment and amount produced short-term effects. The present disclosure can be extended beyond the described single form of donor bacteria and plasmid.

### Methods

To begin examining gene transfer throughout the gut microbiota, we selected a plasmid pWR011 harboring a Kanamycin resistance cassette, ColE1 high copy origin of replication, and a red fluorescent mCherry gene under control of a PL(tetO) promoter. This was transformed into chemically competent E. coli that then served as the carrier bacteria for the subsequent murine studies. The presence of the reporter gene and selectable resistance allowed the bacteria which received and expressed the plasmid to be easily discerned. 16 Swiss-Webster mice (4 weeks of age from Charles River Labs, Wilmington, MA) were used in the studies involving examination of the effect of antibiotic pre-treatment and length of dosing. The mice were separated into 4 groups as follows: no antibiotic pre-treatment+1 week of carrier bacteria; no antibiotic pre-treatment+1 month of carrier bacteria; antibiotic pre-treatment+1 week of carrier bacteria; antibiotic pre-treatment+1 month of carrier bacteria. Antibiotic treatment was provided by replacing the water bottles with the following aqueous solution (1 g/L ampicillin, 1 g/L neomycin sulphate, 1 g/L metronidazole, 0.5 g/L vancomycin, and 5% sucrose). Carrier bacteria treatment was provided by replacing the water bottles with an aqueous solution containing 106 CFU/mL of carrier bacteria containing pWR011. An additional 24 Swiss-Webster mice were separated into 3 groups of 8 (4 male and 4 female) without addition of antibiotic and provided a single day dose of either 2×10⁹, 5×10⁸, or 3×10⁷ CFU of pWR011 carrier bacteria. Fecal pellet samples were collected regularly and plate-based cultures were used to determine the proportion of bacteria possessing the introduced plasmid within samples. The antibiotic and reporter genes allowed for simple quantification of the proportion of pWR011 carrier vs non-carrier bacteria without the plasmid.

### Results

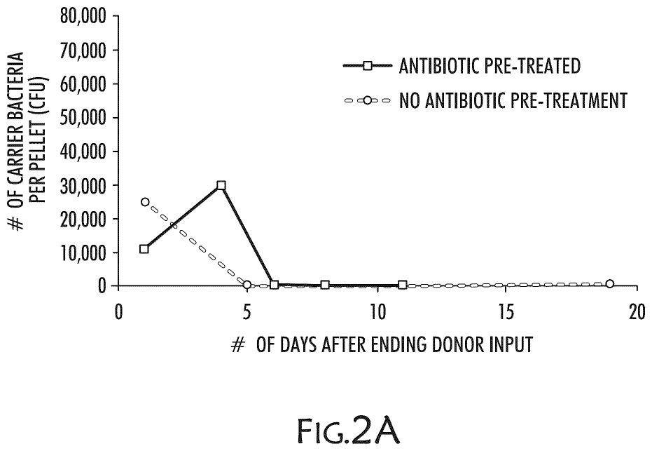

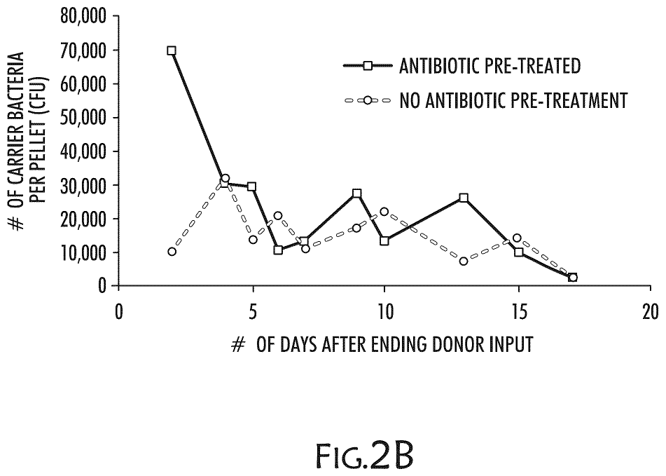

In order to examine the persistence of plasmid carriers in the murine gut, we analyzed the number of colony forming units (CFUs) from fecal pellets selected on Kanamycin containing media. To ensure accuracy in colony counting experiments, sample plating was conducted in sets of 5. The results of our analysis of the fecal pellets bacterial content reveals the number of bacteria possessing the pWR011 plasmid for mice having received 1 week of plasmid donor E. coli (FIG. 2A). Here, we can see the number of carrier bacteria observed for mice having undergone pre-treatment with broad spectrum antibiotic as compared to no antibiotic treatment. The persistence of the plasmid was almost one week after removal of the input dosage of donor bacteria, but was only marginally shorter for the case of no antibiotic treatment.

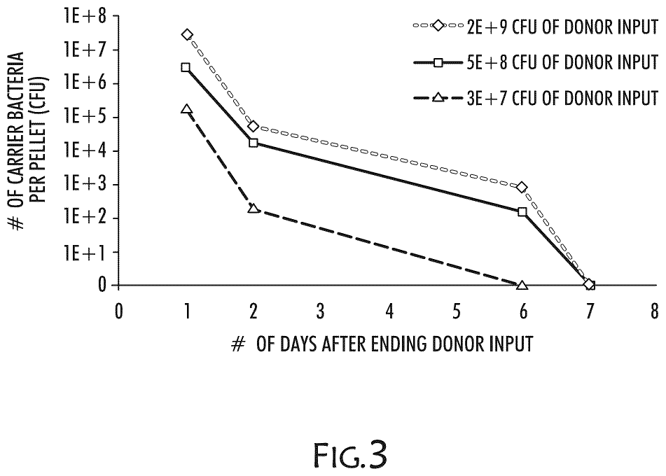

To determine if a longer dosage period for the donor bacteria would result in a change in the observed persistence of plasmid, we examined the presence of carrier bacteria in fecal pellets of mice that received 1 month of plasmid donor E. coli. The results in FIG. 2B show that providing a longer dosage time of 1 month for the plasmid donor E. coli could result in a significantly longer duration of persistence over 2 weeks as compared to less than 1 week if provided a dose of donor bacteria for 1 week. Looking at the quantities of plasmid carrier bacteria observed from both the antibiotic pre-treated and untreated mice after the donor E. coli dosing periods, the abundance appears to persist in the range of 10,000 to 30,000 CFU per pellet. In comparing different single day dosage amounts of pWR011 plasmid donor bacteria (FIG. 3), mice were found to have a larger amount of carrier bacteria present in the fecal pellet relative to a larger initial dosage amount; however, there was a similar extent of one week in which the carrier bacteria persisted.

### DISCUSSION

We found that mice provided a 1-week dosage of donor E. coli are able to provide plasmid carrier persistence for almost a week. Interestingly, we note that the higher the single day dosage amounts of donor bacteria produced increases in carrier persistence but did noticeably provide increased abundance on plasmid carrier. In looking at the longer dosing regimens, we do see a very significant increase in the duration of the persistence of the plasmid within the gut. Not intending to be bound by theory, we believe this to be attributable to the longer duration course of donor bacteria providing more opportunities for transfer of plasmid to the existing commensal gut bacteria. There also exists the possibility that donor E. coli themselves are residing longer within the gut. Our comparison between broad-spectrum antibiotic pre-treated mice and untreated mice did not reveal any long-term differences in persistence; therefore, again not intending to be bound by theory, we believe these results may rule out long-term effects of donor E. coli residence as being the main contributor to the extended duration of persistence observed when using 1 month dosages. A higher carrier bacteria abundance observed for antibiotic pre-treated mice during the donor E. coli dosage period could also be observed and maybe reasonably attributed to increased survival of the input E. coli. Based on observations of decrease in the total commensal gut bacteria density during antibiotic use we may expect corresponding increases in donor survival. It is expected that these observed plasmid carriers counted in the fecal pellets of the 1-week dosage study were from the pWR011 plasmid donor E. coli that had survived the transit through the gut rather than the carriers being only transconjugants.

### CONCLUSION

As disclosed in this Example, we provide an assessment of the plasmid carrier persistence in the murine gut. We revealed the effect of duration in plasmid donor dosing as well as antibiotic pre-treatment on persistence (FIGS. 2A and 2B).

### Example 2

### Controlling the Persistence of an Engineered Bacterial Metabolite Pathway for Isoflavone Conversion in the Gut

### SUMMARY

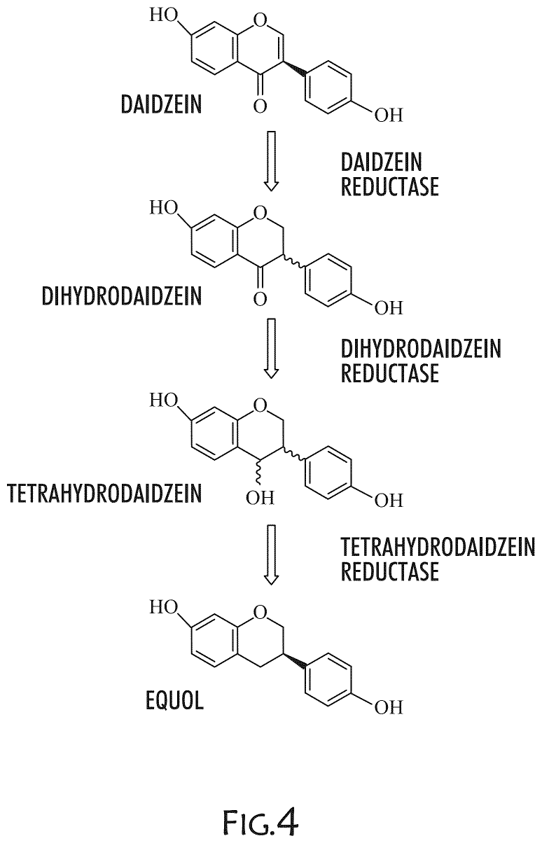

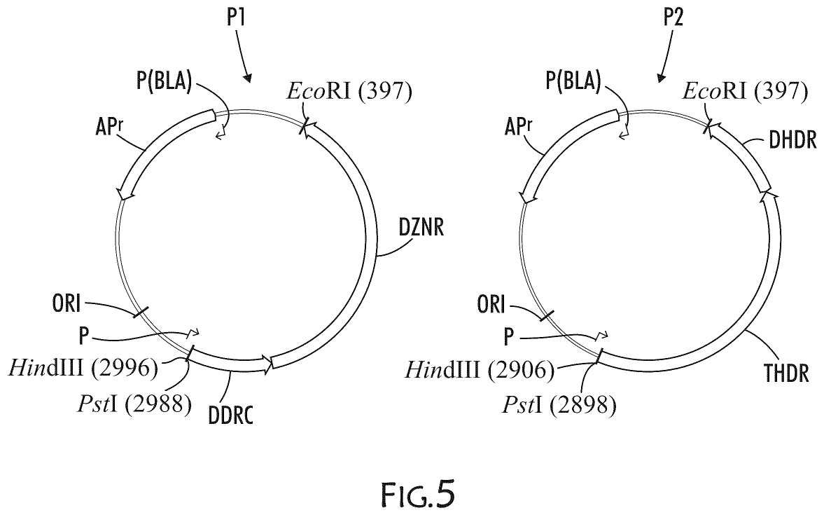

Metabolic pathways provided by bacteria in the gut can change the structure of dietary compounds that are ultimately absorbed by the host and in turn influence health. A natural isoflavone known as Equol is one such case of a microbially synthesized metabolite formed from dietary Daidzein. Equol has been found to be a selective agonist for estrogen receptor beta signaling indicated in reducing large artery stiffness. Unfortunately, only a fraction of the population possesses the intestinal bacteria capable of converting Daidzein from dietary soy into Equol such as via the pathway illustrated in FIG. 4. Such individuals with an Equol producing phenotype experience a range of health benefits with soy intake, including decreased risk of cardiovascular disease. In this Example we describe a means to engineer the intestinal microbiota toward an Equol producing phenotype by introduction of plasmids (FIG. 5) to the gut bacteria for expression of the enzymes Daidzein reductase (DR), dihydrodaidzein reductase (DHDR), and tetrahydrodaidzein reductase (THDR). Here we provide evidence of an engineered bacterial metabolic pathway for enzymatic conversion of the isoflavone Daidzein to Equol, and moreover we describe a strategy for controllable persistence of this engineered genetic construct among the gut bacteria.

### Methods

A set of plasmids was first constructed to facilitate expression of the enzymes DR, DHDR, and THDR from Slackia isoflavoniconvertens which is a recently isolated bacteria from a healthy human gut described in literature to have high activity for Daidzein conversion to Equol. The gene products were expressed in E. coli and the cultures were provided varying concentrations of Daidzein in order monitor its conversion. Production of dihydrodaidzein, tetrahydrodaidzein, and Equol (the desired end product) were assessed qualitatively by thin layer chromatography of the extracted isoflavones and monitored quantitatively by liquid chromatography—mass spectrometry as a function of time. We first administered to Swiss-Webster mice distinct amounts of E. coli carrying a plasmid to express a red fluorescent mCherry reporter. Conditions were varied with respect to the concentration and length of E. coli dosage, and additional experiments using either antibiotic pre-treatment or concurrent treatment were also conducted. Fecal pellets were assessed longitudinally to serve as a representation of the proportion of reporter plasmid carrying bacteria that remained in the gut, and 16s metagenomics analysis was carried out to assess bacterial diversity.

### Results

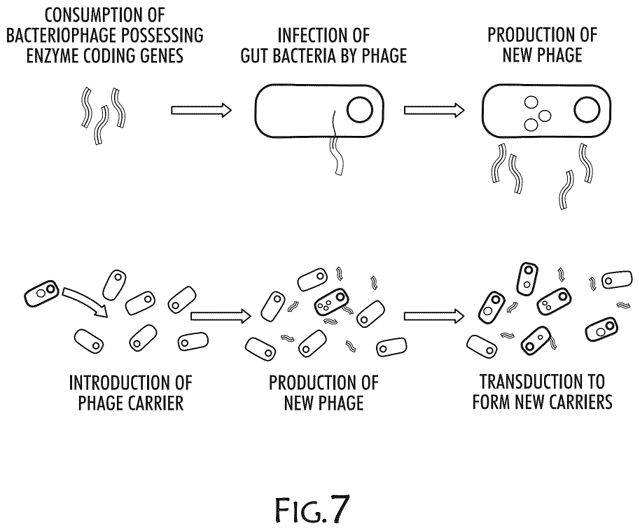

We found that using the P1 plasmid gene products we can convert Daidzein to dihydrodaidzein. Further, p2 plasmid gene products can be used to convert dihydrodaidzein to Equol. Qualitative TLC (FIG. 6) and quantitative LCMS reveal the conversion of Daidzein to Equol using combination of P1 and P2 plasmids expressed gene products. The transfer of genetic constructs to modify gut phenotype is further illustrated in FIG. 7, which shows the infection and transfer of enzyme coding genes to gut bacteria. Persistence of the engineered gene constructs as a function of dosage amount is also described along with the persistence of gene construct as a function of pre-treatment and length of dosage.

### DISCUSSION

Examining the enzymes DR, DHDR, and THDR, we found that these enzymes demonstrated conversion of Daidzein to Equol. In assessing plasmid persistence in the murine gut, we herein describe that the amount of donor bacteria and use of antibiotic pre-treatment marginally influenced the duration of plasmid persistence but did influence short-term abundance. In contrast, providing a concurrent low dosage of antibiotic for which the plasmid carried the corresponding resistance, showed a significant increase in the plasmid persistence which may be attributable to the selective advantage for the carrier bacteria and potentially the resident bacteria as well to retain the plasmid. By altering the dosage duration, we found that we could also effectively modulate the persistence but not to the same degree as that using concurrent antibiotic treatment. We also describe how these dosage strategies may alter compositional diversity of the gut microbiome.

### CONCLUSIONS

In summary, this Example provides a temporal assessment of plasmid introduced to the murine gut, which provides a strategy for controlling plasmid persistence as a function of provided dosage. We also demonstrate the activity of an engineered vector including DR, DHDR, and THDR. The vector once introduced to E. coli can convert Daidzein to the estrogen receptor beta agonist known as Equol. We describe new methods for the introduction and persistence of this metabolic pathway to the gut as one possible approach to engineering the intestinal microbiota toward an Equol producing phenotype in a more socially acceptable alternative to fecal transplant therapy.

### Example 3

### Engineering the Gut Virome for Equol Conversion as a Countermeasure for Arterial Aging

As an extension of Example 2 above and other disclosure herein, we describe herein the engineering of the gut virome for Equol conversion as a countermeasure for arterial aging. Cardiovascular disease (CVD) is the leading cause of death in the industrialized world and advanced age is the major risk factor for CVD. It is projected that by 2030, >40% of the population will have some form of CVD. Vascular aging manifests itself primarily through increases in large artery stiffness and impairments in endothelium dependent dilation (EDD) due to decreased nitric oxide (NO)-bioavailability and increases in reactive oxygen species (ROS). Both large elastic artery stiffness and endothelial dysfunction are predictive of future cardiovascular events and are present in patients with clinical CVD. A recent report has shown that broad spectrum antibiotic treatment to deplete the microbiota improves large artery stiffness and endothelial function in old mice, this was also associated with improvements in ROS. Although this report demonstrates a role for the microbiome, complete depletion of the microbiota as a prevention strategy for CVD is likely unfeasible and other strategies are needed. Equol is a selective agonist for ERbeta unlike other products that can activate ERalpha, androgen receptors, glucocorticoid receptors, progesterone receptor, and mineralocorticoid receptors. Only a minority fraction of the population possesses the proper intestinal bacteria capable of converting Daidzein, an abundant isoflavone in soy based products, into the natural product Equol. Individuals who have gut bacterial strains capable of producing Equol from dietary soy (referred to as Equol producers) experience a range of health benefits with soy intake. For example, Equol-producing postmenopausal women have demonstrated reduced levels of serum LDL-C of 6-8% as well as reduction in C-reactive protein after supplement with dietary soy but this effect is not seen for purified Daidzein supplement. Fecal transplant therapy can be provided to convert non-producers into Equol-producers where the new microbiota provides the ability to metabolize Daidzein to Equol. Thus, the ability to metabolize the Daidzein in dietary soy into Equol may be dependent on each individual's microbiota. Repeated testing of Equol-producers over prolonged periods indicate the status of the microbiota are remarkably stable once becoming an Equol-producer; however, the stigma of fecal transplant therapy along with ethical and social concerns prompt for a more widely acceptable alternative to engineer the intestinal microbiota.

### Example 4

### Engineering E. coli for Conversion of Dietary Isoflavones in the Gut

### INTRODUCTION

Introducing metabolic pathways to the gut is important to tailor the biochemical components ultimately absorbed by the host. Given identical diets, hosts possessing different consortia of gut bacteria can exhibit distinct health outcomes regulated by metabolic capabilities of the gut microbiota. The disparate competency of the population to metabolize isoflavones, such as dietary Daidzein, has shown health benefits for those individuals possessing gut bacteria capable of producing Equol from Daidzein-rich diets. To begin addressing health inequalities due to gut metabolic pathway deficiencies, we developed a probiotic that allows metabolism of isoflavones as to provide a gut phenotype paralleling that of natural Equol producers. Further, we engineered E. coli to produce the enzymes necessary for conversion of Daidzein to Equol, and as demonstrated in a murine model these bacteria enabled elevated serum Equol levels to dietary Daidzein.

Isoflavones and isoflavone-derived compounds with structural similarity to steroid hormones can bind to estrogen or androgen receptors within different tissues serving as agonist or antagonists. Because dietary isoflavones can undergo different forms of biotransformation by gut bacteria, the metabolic pathways present within the gut can influence host health by modulating the biochemical profile of absorbed compounds. It has been reported that a minor fraction of the population possess gut bacteria capable of metabolizing dietary Daidzein into the estrogen receptor beta agonist known as Equol. Individuals displaying an Equol producing gut phenotype however have demonstrated health benefits, including reduced risk of cardiovascular disease when consuming Daidzein-rich foods such as soy. Accounts of microbial cultures derived from human fecal samples have shown Equol-producing capability can be imparted upon mixing of those bacteria with fecal culture from individuals that were not Equol producers. Providing a host with exogenous gut bacteria that express the enzymes needed for conversion of Daidzein into Equol has been shown in fecal transplant therapy to achieve an Equol producing phenotype in the host gut. In this Example, we describe development of a probiotic strain of E. coli that can provide conversion of dietary Daidzein into Equol to serve as a means for introducing the metabolic pathways necessary for the host gut to attain an Equol producing phenotype. The significance of providing the host with the ability to convert Daidzein to Equol follows from intestinal absorption of the Equol product, since increased serum Equol has reported positive endocrine-related health effects as a selective estrogen receptor modulator.

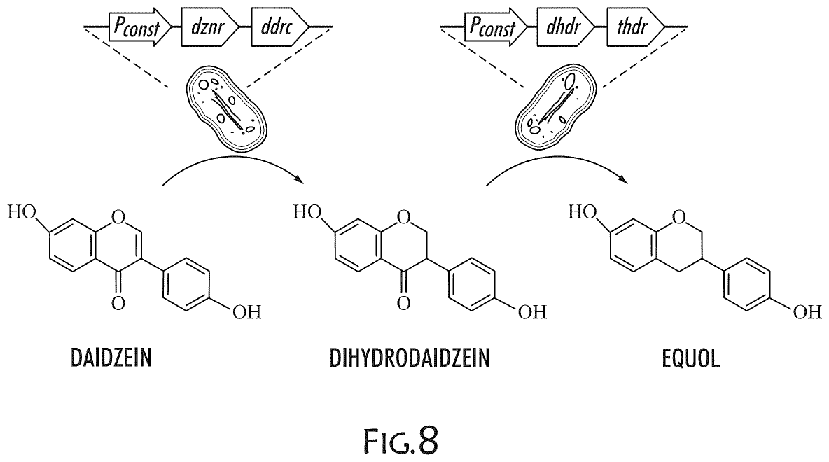

We describe the use of two separate engineered probiotic strains of E. coli Nissle 1917 for compartmentalizing the DZNR and DHDR enzymes in separate cells (FIG. 8) to mitigate inherent metabolic burden of expression and NADPH cofactor demand. Here we utilized the metabolic pathway from a non-Coriobacteriaceae strain, specifically the Lactococcus garvieae 20-92 strain, known to facilitate complete Daidzein to Equol conversion. The genes from L. garvieae 20-92 show 99% similarity to those of Coriobacteriaceae and are believed to be a result of horizontal transfer from Coriobacteriaceae. Here we engineered two probiotic E. coli Nissle 1917 strains by transformation with one of our constructed plasmids, either P1-DZNR-DDRC (for expression of the DNZR and DDRC gene products of L. garvieae) or P2-DHDR-THDR (for expression of the DHDR and THDR gene products of L. garvieae) as depicted in FIG. 8. In the present disclosure we report the first expression of the enzymatic pathway for conversion of Daidzein to Equol in E. coli Nissle. Our studies examining the persistence of E. coli Nissle 1917 within the murine gut suggest a twice weekly supplement of bacteria at 10⁸ CFU/mL to be sufficient for maintaining the presence of the engineered E. coli Nissle without the use of selection pressure. Thus, we examined the ability of the combination of engineered E. coli Nissle strains harboring P1-DZNR-DDRC or P2-DHDR-THDR, herein referred to as Nissle P1 and Nissle P2 respectively, to provide production of Equol within the gut when co-supplemented along with dietary Daidzein in murine studies. As discussed below, we confirmed the conversion of Daidzein to Equol in cultures of Nissle P1 mixed with Nissle P2 as well as examined the individual whole-cell biotransformation rates in separate cultures. Moreover, we present below our findings from in vivo murine studies showing that administration of Nissle P1 and Nissle P2 to mice results in enhancement of serum Equol levels when provided dietary soy.

### Results and Discussion

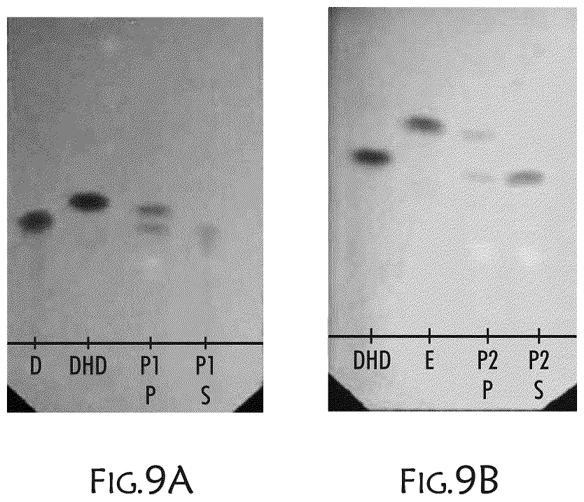

In this Example, the probiotic E. coli Nissle 1917 strain was engineered to express these recombinant proteins of L. garvieae strain 20-92, wherein two separate E. coli Nissle strains were produced. Specifically, transformation of E. coli Nissle with plasmid P1-DZNR-DDRC was carried out to produce the DZNR and DDRC expressing strain Nissle P1 and similarly transformation with P2-DHDR-THDR was used to produce Nissle P2 which expresses the DHDR and THDR genes. The separation of these genes across two strains is notable and is related to the NADPH cofactor requirements for catalysis by both the DZNR gene product and the DHDR gene product, to alleviate competition for NADPH and reduce the burden of overexpression of multiple recombinant enzymes in a single host cell. Using this approach, the formation of dihydrodaidzein product by Nissle P1 and Equol product by Nissle P2 was confirmed by thin layer chromatography (TLC) (FIG. 9A).

As shown in the TLC images, Daidzein control (lane 1), dihydrodaidzein control (lane 2), extracted sample of cell pellet from Nissle P1 culture provided Daidzein (lane 3), and extracted sample of supernatant from Nissle P1 culture provided Daidzein (lane 4) showing that the enzymes expressed in Nissle P1 remained within the cells and were not released into the supernatant as only the cell pellet sample showed conversion of Daidzein to dihydrodaidzein. B) TLC of dihydrodaidzein control (lane 1), Equol control (lane 2), extracted sample of cell pellet from Nissle P2 culture provided dihydrodaidzein (lane 3), and extracted sample of supernatant from Nissle P1 culture provided dihydrodaidzein (lane 4) showing that the enzymes expressed in Nissle P2 remained within the cells and were not released into the supernatant as only the cell pellet sample showed conversion of dihydrodaidzein to Equol

The activity of each strain was initially confirmed by TLC of the extracted product of Nissle P1 and Nissle P2 cell culture in the presence of Daidzein and dihydrodaidzein substrate, respectively. As seen in FIG. 9A, the use of the whole-cell reaction system of Nissle P1 when provided 90 μM of Daidzein substrate revealed conversion to dihydrodaidzein as shown by TLC comparison with standards. Here we see that the conversion was carried out within the cells rather than secretion of enzyme for extracellular reactions as addition of substrate to the supernatant sample did not yield any noticeable conversion to product. Similarly, TLC comparison of standards of dihydrodaidzein and Equol to samples of Nissle P2 whole-cell reactions with dihydrodaidzein substrate showed the Nissle P2 ability to convert dihydrodaidzein to Equol (FIG. 9B). The results show these reactions are taking place intracellularly as no reaction products were formed when introducing the substrates to the cell culture supernatant.

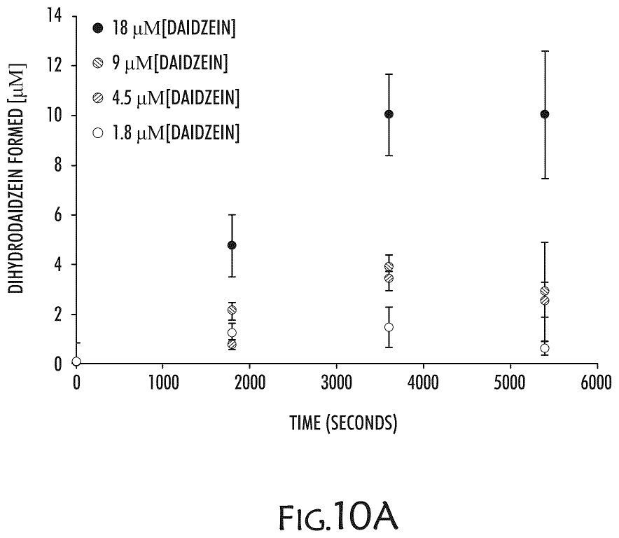

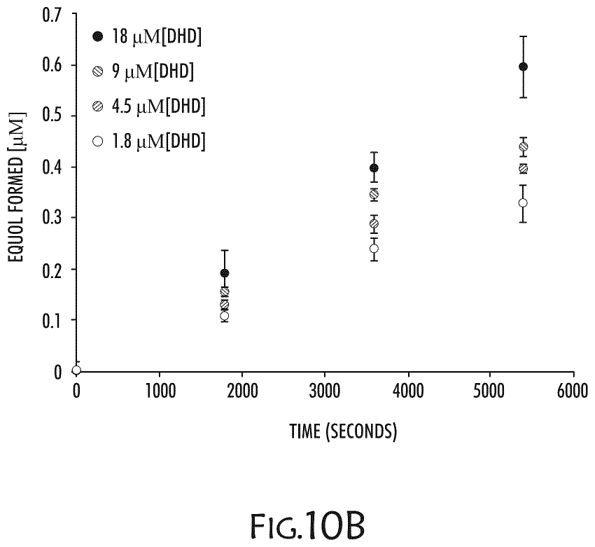

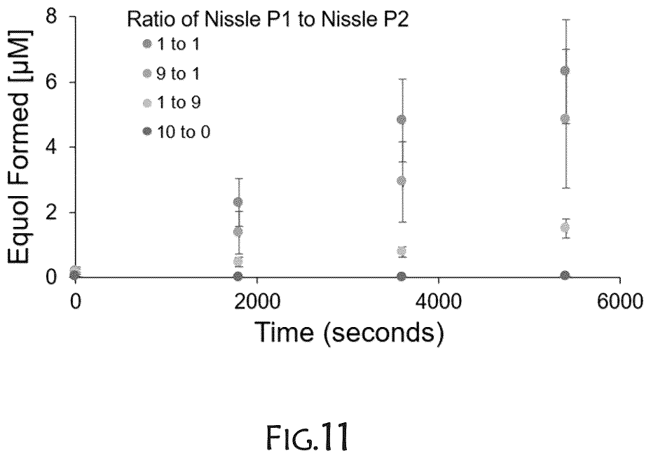

To identify which, if either, of the strains in the two-strain conversion system was rate limiting, we investigated the amount of dihydrodaidzein and Equol production for whole-cell reactions of Nissle P1 and Nissle P2, respectively. As shown in FIGS. 9A and 9B, the rate of Nissle P1 conversion of Daidzein to dihydrodaidzein was concomitant with substrate Daidzein concentration as quantified by liquid chromatography mass spectroscopy (LCMS). Nissle P2 rates of conversion of dihydrodaidzein to Equol were examined as well and also found to increase with substrate concentration as shown in FIGS. 10A and 10B. More particularly, FIG. 10A displays conversion of Daidzein to dihydrodaidzein by whole-cell culture with Nissle P1, and FIG. 10B displays conversion of dihydrodaidzein to Equol by whole-cell culture with Nissle P2.

From these whole-cell kinetics experiments, we could identify the projected maximum rate for Nissle P1 formation of dihydrodaidzein (˜1.8 nM/s) as compared to the projected maximum rate for Nissle P2 formation of Equol (˜0.1 nM/s) revealing an order of magnitude faster rate for product formation by P1 wherein the projected maximum rate of product formation was determined based on a Michaelis-Menten model of kinetics. The enzyme was not found to be vulnerable to oxygen with little to no activity under aeration (shaking) conditions. Our reactions which were all conducted under aerobic conditions with orbital shaking at 180 rpm

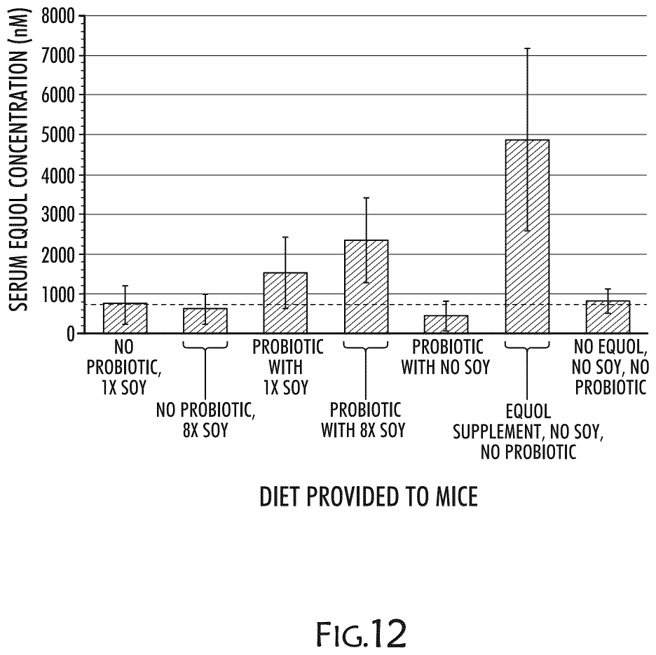

Examining a mixed cell biotransformation system in which Nissle P1 and Nissle P2 were cultured separate and then mixed to provide a two-strain whole-cell reaction showed the final Equol product was formed upon addition of Daidzein substrate to the culture. Mixed cell populations of Nissle P1 and Nissle P2 at different proportions of Nissle P1 to Nissle P2 and holding the total amount of cells in the culture constant revealed that the ratio of the strains impacted Equol production as shown in FIG. 12. Specifically, with increasing amounts of Nissle P2 in the mixture we observed an increase in the rate of Equol production until a point at which a higher amount of Nissle P2 than Nissle P1 was used wherein we observed a decrease in the rate of Equol production. Not intending to be bound by theory, this may be attributed to compartmentalization in our system as the impact of spatial separation on the dynamics of multi-cell processes has been investigated and can be influenced by the rates of product release, rates of substrate uptake, diffusion, and concentration dependent uptake factors.

Interestingly, we see that the substrate flexibility of the L. garvieae 20-92 enzymatic pathway allowed for conversion of genistein to 5-hydroxy-Equol wherein we also observed an analogous faster rate in processing genistein to dihydrogenistein by Nissle P1 as compared to Nissle P2 showing slower formation of 5-hydroxy-Equol. This shows that the second half of the enzymatic pathway (that being dihydrogenistein to 5-hydroxy-Equol) proceeds slower than dihydrogenistein formation from genistein.

In order to demonstrate the ability of the Nissle strains P1 and P2 to be utilized as a cooperative mixture of probiotics to enhance the metabolic gut pathways for conversion of dietary Daidzein into Equol, we carried out a study in mice examining the serum Equol levels as a function of dietary Daidzein in the form of soy powder and supplementation of probiotic Nissle P1 and Nissle P2 via drinking water. Serum Equol levels of the mice were measured after 1 week of consumption of a low or high soy content supplement (1× or 8× soy loaded hydrogel). As seen in FIG. 12, serum Equol levels of mice provided soy were at the quantitative lower limit of detection of the assay. In contrast, providing mice with the 1× or 8× soy hydrogel along with simultaneously co-administration of Nissle P1 and Nissle P2 resulted in increased serum levels of Equol relative to the amount of soy provided within the hydrogel with serum Equol levels reaching nearly 2.5 μM for mice provided the high soy content hydrogel. Two sets of negative controls were provided as reference. In the first negative control, mice did not receive soy but did receive the two Nissle strains, while in the second negative control group mice did not receive soy and also did not receive the two Nissle strains. For each of these negative controls, the Equol level remained at the detection limit indicating no intrinsically elevated Equol levels due to the probiotic alone or due to the diet alone. In addition, a positive control group consumed Equol that was supplement directly in the hydrogel to reveal, as expected, a very high elevation in serum Equol concentration reaching almost 5 μM without the addition of the engineered Nissle P1 and Nissle P2 strains. The results from these experiments with Nissle strains P1 and P2 represent the first in vivo demonstration of consuming recombinant strains of E. coli for conversion of dietary Daidzein to Equol in the gut to result in elevated serum Equol levels.

To summarize, the examination of the co-administered Nissle P1 and Nissle P2 as probiotic for mice consuming Daidzein-rich soy revealed an impressive increase in the serum level of Equol present as compared to mice receiving no probiotic. The effect was more pronounced for mice provided supplement containing higher concentrations of soy. These levels provide one possible means for imparting a host gut with an Equol producing phenotype under a Daidzein-rich diet. In conclusion, we have demonstrated the successful use of recombinant genes from L. garvieae strain 20-92 expressed in E. coli Nissle for the conversion of the isoflavones Daidzein and Genistein. Finally, this two strain compartmentalized system demonstrated effective conversion of dietary Daidzein within a host murine gut after consumption of soy to form the product Equol resulting in elevated serum Equol levels.

SEQUENCES

Sequence definitions
DZNR sequence (daidzein reductase [Lactococcus garvieae]):
ATGAAGAATAAATTTTATCCCAAAACCTTTGAACGTGGTTAC
ATCGGGAATTTGGAAGTAGAAAACCGCGCGATCCGCATGCCTATGGGAAC
AGAACTTGGCAACCCCGACGGTTCGCCCTCATGGGCTTCCCTTAAGGCGTA
TGCTGAGGCTGCGGATGGTGGGACAGGTATTGTCTTTATGGATAACGCAG
GTGTTACTCAGTTCCACCATGTCGGTTTGTCGCTGGCGTCAGATAATTATAT
CGGTCCTATGAGTGTCTTGGCGAAAACTATTAAGCAGCATGGGGCAATTCC
AGGTCTTCAAATTGTGCACCCTGGACGTGATGCTGCATTTGTGCGTGGCGA
CGATCTTATCAGTTCTAGCCGCATTCAGTGGGAGCCTTGGTATGAAAATGG
AGGGGCTGTTCCTCGCGAGCTGACAATCGAGGAAATTCATGATTTTGTGGG
GTACTTCGGTGATTGTGCTTTACGTGCGCAAACAGCGGGGTTCGAAATCGT
TGATGTCCACGCGGCGTGCGGGGTCCTGTTGTCCAACTTCTTGTCACCTCG
CAACAATACTCGTAACGATATGTATGGCGGCTCGTTGCACAATCGTGCACG
CTTTTTGTTAGAGGTAATCCGCGATATCAAAAAGAAGTGCCCGAACCTTCC
ATTAGCTATTCGTCTTAGTGGAATCGACTTCGAGCCCGATGGAATCACTAT
CGAAGAGACATGTGAGGTCGCGAAAATGTGTGAAGCAGCTGGGGCTGACG
CCATCAACATTACCTGGGGCTCTCATGCTGAGGTAATCAATGCAGCAGGTT
TGCTTTCTAAACATGGAGCGAACCACGTCGAGGCCGCGAAAATGATCAAA
GACGCGGTAAGCATCCCGACTATGTTATGTGGGGGTATCTACAGCCCAGA
AATTGGCGAAAAGTTACTTGAGGATGGTGTCTGCGACTTCATCGGGATTGG
TAAGCCAGCTCTGGCTGACCCCATGTGGGCTAAAAAGGCGGCTGAGGGAC
GCCCCGAGGACATTCGTCCGTGCATCGGTTGTGGAGTTGGATGCCACGATC
GCGGGATGCTTTCCGGTGGAGTGGTACAATGCGCAGTCAACGCTGCCCTGT
ACAAGTTTGATGAACCTGTCTACCCTCAAGCGGAAGTTCCCAAGAAAGTTA
TTATTATTGGAGCGGGACCGGCAGGTTGTGAAGCAGCCATTACAGCGAAA
AAATGCGGGCACGACGTCACAATTTACGAGAAGCGTAAAATCGGTGGCGT
GCTGAAGGAGGCCACTGTGAGCGACTCCAAGGAGGATCTGGGTCGCTTAA
TCACATATTACGAGACCCAGTTGAAAAAGGAGGGAATCGAGGTAATCTAT
GAAGAAGCGACCGCGGATACCGTAGTGGCGGGCGGTTTCGACGTTGCGAT
TGTTGCGTGTGGAGCCACAGTTCGCAATCTTAACATTGACGGCCAAGACGA
CCCAAGCGTTGTGTACGCTATGGACTTCCTGGATAACGATTGCAAGTCAGA
CGCCGACCGTGTAGTAGTTGTCGGGGGCGGAATTGTTGGCGCGGAAACTG
CGCTTATTCTTGCGGAGGAGCGTGGGAAGGACGTAACCATTACAACTCGCT
CACCAGAGTTCTTTGTATCTGGAGTGATGGGGATTGCCTATATGGTACGTT
TAGGCATGGCCGGTGTAACCATTAAGCCCAGTACTCAATTGGTCGCCGTTA
AGGATGGAAAGCCGATGTTCGCCGGGCCCCGTGGTTTGGAAACTCTTGAT
GTGGACCAGACTATCATCTCTAGTGGCTTCGTGCCCACGTTTAACCAATTC
CGTGCACAGATTGAAGAAAAGTGCGAAGACGTGCGTGTGATCGGGATCGG
CGATTGCAAAGCATCTCGTATGGTAATGGATGCCGTTCATGAGGGGTACAT
CGCAGGCTGTAACCTGTAATAG. (SEQ ID NO. 1).
DDRC sequence (dihydrodaidzein racemase [Lactococcus garvieae]):
ATGATTAAAGCACAATTAAATCGCATTGCGTTACGCGCTGCC
GATGCTGATAAAGCCGTCGAAGACCTTAACAAATTGTTAGGTGTAACCTTC
TACGGGCCGTACGATGACGAACACATGGGCCTGCGCGTTGCGTTACCGAA
ATCGGGGGGCATTGAAGTCATGGCCCCGATGCACGATCACGACGCCATCG
GCGCATATAAGGCATTACAGACGGTCGGAGAGGGTATCAGTGGTATCGCA
ATGCGCGTGGACGACTTTGATGCCGCCAAGGAACACTTCGCAAGTCTTGG
CTTAACGCCGGTTGTAGAGTTCAATCACGGAAAGTTTCGTGAGATGATCTT
TCCCCCTCAGCCGGCAACGCACAACCTGGAAATCGCTGTGAACGAATTTCC
AGATGCTAACGGCGCGGCCATTCAGGTGGCATTAGATATGGGTGTAGACT
GGACTGATGTTTGTGACTGGGATGCGAAGTAGTAA. (SEQ ID NO. 2).
DHDR sequence (dihydrodaidzein reductase [Lactococcus garvieae]):
ATGGCTCAGGAAGTGAAAGTACCAAAAATGCCCGGTGCACC
TGTTTTTGGTAAATGGATCTCACCGGAGGAGTCCGTGGGTCAGCGCCTGAA
GGGTAAGAAGATTTTGTTGACCGGGACTACTAAGGGGGTGGGCCGTGTAA
CGCAAGAGTTACTGTGTGCACATGGAGCGTTCGTGTGTGGGAGTGGCCGC
ACGCCAGGGGTGGCTGCATCAGTTGCCGACGAATTGAAAGCAAAGGGTTA
TCAGGCCGCAGGAATGGATGTAGACCTTTCTGACTACGATGCTGTAAAAA
AGTGGGTCGAGGAGTGCGCCGAACTGATGGGTGGAATTGATGTTGTGATC
AACAATGCCTCACACCCTGGCATGGCACCCTTCGGTGAGATGACACCGGA
AATCTGGAATTACGGAATCAAGAACGAGCTGGATTTAGTGTACAACGTAT
GCAATTGTGCATGGCCGTATTTACAAAAAGCCGATGGGGCATCAATCATTA
TTACGTCAAGCACCGTAGCGTTACAAGGGAGCAACTCACCTCAGGCATGT
CACGCAGCTTGTAAAGGGGCCTGTCTTTCACTGGCACGTCAGCTTGCCGCG
GAAGGTGGGCCATTCGGAATCCGCTGTAACTCGGTTACGCCCGGCCTTGTT
TGGACTGAGGCAATGAGTAACATCCCGAAGGAAATGGCCTCTGGTTTGGT
TGCCGCTCAAACAACTCAGCAGGCCGTTGACCCGATGGACATTGCTTACGC
CTATCTGTTCTTAGCCTCTGACGAAAGTCGCCAAATTACTGCCGCGAATAT
CCCTGTGGATGGAGGTTGTGCAGGTGCCGTAACCGGTGGAATGCAGGGCG
AAATCGAAGTATAATAG. (SEQ ID NO. 3).
THDR sequence (tetrahydrodaidzein reductase [Lactococcus garvieae]):
ATGGCGGAGTTTGACGTAGAGTATGATTTGGTAGTCGTCGGC
GGGGGGGCTTCCGGCAAATCAGCTGCCTTGATTGCTGCCCGCGAAGGGAA
ACGTGTTGTCGTTTTGGAGAAGATGCCGGAGACGGGGGGTTTGTCGATGTA
CGCCGAGGGCACAGCAGCCTTTGAGTCTAGCATTCAGAACGAATTGGGAA
CTCCCCGCCTTAGTAAATATCACTTTCCCACGAAACAGGAAGGCATCGAA
AAGTTTATGGGGTATTCGCATCAACGTGCTAATTACGACGTAGTTCGTGCC
TTCGTCGAAAATAGCGCCGAAACCATCGACATCTACCGTGACCTGGGTGT
AGTATATAAAGCATGCGACATCGCCGCTGAAGACGATCCAAACGAGGTCT
GGACGTTTCACTTGCCTGAGGGTCTTGGAGCACATTGTCAGGAAGTTCTGC
TTGACGCGATTCAAAAACTGGATGTTGATATTTTCACATCCACTCCGGCGA
AGGAACTTATCATTGAAGACGGGGCTGTCGTAGGAGTCGTCGCTGAATCC
GACGGCGAGCCTCTGCGTGTGGGTGGCAAAGCTGTCATCCTTGCGACCGG
AGGTATGGGATCAAGTCCAGAACGTATCTTCAAATATTCTTGGTTTGCGCC
CGCGGCATATAATATGAACACTTTAACACCGCTTCAGAACGTAGGGGATG
GCTTAGACCTTGCACTGTCAGCCGGGGCCGACCCGACCTACATCACTACTT
GTCCAATCCTGGCAGCAGGAGGCCGCGACATGACTATGGACAGCCAGGTG
GGAGGTGCCGGTGTTAATCCGGGGGTGTGGATTAACAAAACGGGACGCCG
TTTTGCGGCCGAATCAGTAGCGGAGAATATCGGGGACATTGGAACATATT
ATGGGAAACAGCCGGGTGGAGTTGTGTGGAGCATTTTGTCTCAGGCGGAC
ATCGATCGCTTGGTCGCCGAAGGCTCTGAGATTGCCATTGGAGAGTTTGTT
GTTTACCATAAGCCTATGGAACGCCTGCCCATCGAGCTGGAGGCCCACCTG
GAGAGCGGGTTAGTTAAAAAGGCGGGAAGTTTTGAAGAACTTGCCGCTTT
AATTGACGTACCCGTCGACACTTTCGTTGCAACAATGGCAGATTATAACGA
GGCGTGTGAGAAAGGGTATGATGATGCTTTTATGAAGAAACCCCAGTACT
TGCGCCCCATGGTTGAAGGTCCGTTCTATGCTATCCCACTTGCAACCGGAA
CCATGGGATCGGCCGGAGGTATTCGCATCAACGGAAATATGCAAGTCGTA
GACGCAGATTATAATGCGATTCCAGGTCTTTATGCCGTAGGGTTGGACGCG
ACCGGTTTGTACGGTGACAGTTACAATATGGAAGTTCCGGGGGCCGCCAA
TGGGTTCGCTCATACGTCAGGACGTATTGCAGCTCGTCACGCTATCTCCAC
GATGTAGTAA. (SEQ ID NO. 4).
Promoter:
TTTACGGCTAGCTCAGTCCTAGGTACAATGCTAGC. (SEQ ID
NO. 5).
BioBrick Scar:
TACTAGAG. (SEQ ID NO. 6).
Ribosome Binding Site:
AAGAAGGAGATATACC. (SEQ ID NO. 7).
Transcriptional Terminator:
CTAGCATAACCCCTTGGGGCCTCTAAACGGGTCTTGAGGGGT
TTTTTG. (SEQ ID NO. 8).
Original vector sequence including a tetA region that was removed
during cloning:
aaagaacgtggactccaacgtcaaagggcgaaaaaccgtctatcagggcgatggcccactacgtga
accatcacccaaatcaagtttttggaggtcgaggtgccgtaaagcactaaatcggaaccctaaagggatctcgggaaaagc
gttggtgaccaaaggtgccttttatcatcactttaaaaataaaaaacaattactcagtgcctgttataagcagcaattaattatgat
tgatgcctacatcacaacaaaaactgatttaacaaatggttggtctgccttagaaagtatatttgaacattatcttgattatattatt
gataataataaaaaccttatccctatccaagaagtgatgcctatcattggttggaatgaacttgaaaaaattagccttgaatacat
tactggtaaggtaaacgccattgtcagcaaattgatccaagagaaccaacttaaagcttatgatagtgatgtgcttaaaaactta
ctcaatggctggtttatgcatatcgcaatacatgcgaaaaacctaaaagagcttgccgataaaaaaggccaatttattgctattt
accgcggctttttattgagcttgaaagataaataaaatagataggttttatttgaagctaaatcttctttatcgtaaaaaatgccctc
ttgggttatcaagagggtcattatatttcgcggaataacatcatttggtgacgaaataactaagcacttgtctcctgtttactcccc
tgagcttgaggggttaacatgaaggtcatcgatagcaggataataatacagtaaaacgctaaaccaataatccaaatccagc
catcccaaattggtagtgaatgattataaataacagtaaacagtaatgggccaataacaccggttgcattggtaaggctcacc
aataatccctgtaaagcaccttgctcatgactctttgtttggatagacatcactccctgtaatgcaggtaaagcgatcccaccac
cagccaataaaattaaaacagggaaatctaaccaaccttcagatataaacgctaaaaaggcaaatgcactactatctgcaata
aattcgagcagtactgccgttttttcgccccatttagtggctattcttcctgccacaaaggcttggaatactgagtgtaaaagac
caagacccgctaatgaaaagccaaccatcatgctattccatccaaaacgattttcggtaaatagcacccacaccgttgcggg
aatttggcctatcaattgcgctgaaaaataaataatcaacaaaatgggcatcgttttaaataaagtgatgtataccgaatt
cgattgcgtctcaacccctacttcggtatctgtattatcacgtgtatttttggtttcacggaaccaaaacataaccacaaggaaa
gtgacaatatttagcaacgcagcgataaaaaagggactatgcggtgaaatctctcctgcaaaaccaccaataataggcccc
gctattaaaccaagcccaaaacttgcccctaaccaaccgaaccacttcacgcgttgagaagctgaggtggtatcggcaatg
accgatgccgcgacagccccagtagctcctgtgatccctgaaagcaaacggcctaaatacagcatccaaagcgcacttga
aaaagccagcaataagtaatccagcgatgcgcctattaatgacaacaacagcactgggcgccgaccaaatcggtcagaca
tttttccaagccaaggagcaaagataacctgcattaacgcataaagtgcaagcaatacgccaaagtggttagcgatatcttcc
gaagcaataaattcacgtaataacgttggcaagactggcatgataaggccaatccccatggcatcgagtaacgtaattacca
atgcgatctttgtcgaactattcatttcacttttctctatcactgatagggagtggtaaaataactctatcaatgatagagtgtcaac
aaaaattaggaattaatgatgtctagattagataaaagtaaagtgattaacagcgcattagagctgcttaatgaggtcggaatc
gaaggtttaacaacccgtaaactcgcccagaagctaggtgtagagcagcctacattgtattggcatgtaaaaaataagcggg
ctttgctcgacgccttagccattgagatgttagataggcaccatactcacttttgccctttagaaggggaaagctggcaagattt
tttacgtaataacgctaaaagttttagatgtgctttactaagtcatcgcgatggagcaaaagtacatttaggtacacggcctaca
gaaaaacagtatgaaactctcgaaaatcaattagcctttttatgccaacaaggtttttcactagagaatgcattatatgcactcag
cgctgtggggcattttactttaggttgcgtattggaagatcaagagcatcaagtcgctaaagaagaaagggaaacacctacta
ctgatagtatgccgccattattacgacaagctatcgaattatttgatcaccaaggtgcagagccagccttcttattcggccttga
attgatcatatgcggattagaaaaacaacttaaatgtgaaagtgggtcttaaaagcagcataacctttttccgtgatggtaacttc
acggtaaccaagatgtcgagttaaccaccctttagattcataaagcgaaaataatgcggctccaacgtacccacctaaatgg
aaacggcgttcactccaatctaaacacgcacaacagattttacgtgaatgtttggaaggaacgtcaattcccatttcatgaaaa
tattgaataccacttaatgtgatcattgaaccattttcagtgatccattgctgttgacaaagggaatcatagatcccccgatttaga
gcttgacggggaaagccggagaacgtggcgagaaaggaagggaagaaagcgaaaggagcgggcgctagggcgctg
gcaagtgtagcggtcacgctgcgcgtaaccaccacacccgccgcgcttaatgcgccgctacagggcgcgtactatggttg
ctttgacgagcacgtacaacgtgctttcctcgttagaatcagaacgggagctaaacaggaggccgattaaagggattttaga
caggaacggcacaccagaatcttgagaagtgtttttgtaatcagtgaggccaccgagcaaaagagtctgaccatcacgcaa
attaaccgttgtcgcaatacttctttgattagtaataacatcacttgcctgagtagaagaactcaaactatcggccttactggttat
atctaaaacaatattaccgccagccattgcaacgggaaaaacactcatagaaataccaacattttgacgctcaaccgtctgaa
atggattatttacattggcagattcaccagttacacgaccagtaataaaagggacattctggccaacagaaatagaacccttct
gacctgaaagcgtaagaatacgaggcacagacaatatttttgaatggctattagtctttaatgcgcgaactgatagccctaaaa
catcgccgttaaaaataccgaacgaaccacccgcagaagataaaacagaggttagacggtcagtattaacaccaccagca
acagtgccgcgctgagagccagcagcaaaggaaaaatctaaagcatcaccttgctgaacctcgaaaattaatccttcaatca
atatctggtcagttggcaaatcaacagtagaaagaaattgcggaaggttatctaaaatatctttaggggcgctaactactaata
agttagaaccatcaacagataatacatttgaggatttagatgtattagatttaacaaacaattctacaacccttattaaatcctttgc
gcgaacgttattaattttaaatgtttgagtaacattatcattttgcggaacaaagaaaccaccagaaggagcggaattatcatca
tattcctgaatatcagatgatggcaattcatcaatataatcctgactatttgggttatatttctgaattatggaaggaattgagccaa
ccatatcaaaattattagcacgtaaaacagagataaagaaattgcgtaaattttcaggcttaacgtcagaggaatatacagtca
ctgtacctttaacatcaggtgagacaataacagattcacctgtttgctttgaataccaagtcacgaaatcgcgcagaggcgaat
tattcatttcaattacttgagcaaaagaagatgatgaaacaaacatcaagaaaacaaaattaattacatttaacaatttcatttgaa
ttaccttttttaattgaaacagtacataaatcaatatatgtgatggaataaccttgcttctgtaaatcgtcgctattaattaattttc
ccttagaatccttgaaaacatagcgatagcttagattaagacgctgagaagagtcaatagtgaatttatcaaaatcataggtctgag
agactacctttttaacctccggcttaggttgggttatataactatatgtaaatgctgatgcaaatcctatcgcaagacaaagaac
gcgagaaaactttttcaaatatattttagttaatttcatcttctgacctaaatttaatggtttgaaataccgaccgtgtgataaataag
gggttaaatatgaataaacacctgaatcataattactggaaaaagcctgtttagtgtcatatgcgttatataaattcttaccagtat
aaagccaacgctcaacagtagggcttaattgagaatcaccatatttaacaacaccaacatgtaatttaggcagaggcatttttg
agccagtaacaagagaatataaagtgccgacaaagggtaaagtaattctgtccagacggcgacaataaacaacgtgttcag
ctaatgcagaacgcgcctgtttatcaacaatagataaatcctgaacaagaaaaataatatcccatcccaatttacgagcatgaa
gaaaccaatcaataatcggctgtctttccttgtcattccatgaacgggtattaaaccaagtaccgcattcatcaagaacaagca
aaccgtttttattttcgtcgtaggaatcattaccacgaccaatagcaagcaaatcagaaatagaaggcttatccggtattctaag
aacgcgaggcgttttagcgaacctcccgacttgcgggaggttttgaagccttaaatcaagattagttgctattttgcacccagc
tacaattttatcctgaatcttaccaacgctaacgagcgtctttccagagcctaatttgccagttacaaaataaacagccatatttat
ttatcccaatccaaataagaaacgattttttgtttaacgtcaaaaatgaaaatagcagcctttacagagagaataacataaaaac
agggaagcgcattagacgggagaattaactgaacgccctgaacaaaatcagagggtaattgtgcgctaatatcagagagat
aacccacaagaattgagttaagcccaataataagagcaagaaacaatgaaatagcaatagctatcttaccgaagcccttttta
aggaaagtaagcagatagccgaacaaagttaccagaaggaaaccgaggaaacgcaataataacggaatacccaaaaga
actggcatgattaagactccttattacgcagtatgttagcaaacgtcgaaaatacatacataaaggtggcaacatataaaagaa
acgcaaagacaccacggaataagtttattttgtcacaatcaatagaaaattcatatggtttaccagcgccaaagacataaggg
cgacattcaaccgactgaggcaaagaaggtaaatattgacggaaattattcattaaaggtgaattatcaccgtcaccgacttg
agccatttgggaattagagccagcaaaatcaccagtagcaccattaccattagcaaggccggaaacgtcaccaatgaaacc
atcgatagcagcaccgtaatcagtagcgacagaatcaagtttgcctttagcgtcagactgtagcgcgttttcatcggcattttc
ggtcatagcccccttattagcgtttgccattttttcataatcaaaatcaccggaaccggagccgccaccggaaccgccaccct
cagagccgccaccctcagaaccgccaccctcagagccgccaccctcagagccgccaccagaaccaccaccagagccg
ccgccagcattgacaggaggttgaggcaggtcagacgattggccttgatattcacaaacgaatggatcctcattaaagccag
aatggaaagcgcagtctctgaatttaccgttccagtaagcgtcatacatggcttttgatgatacaggagtgtactggtaataagt
tttaacggggtcagtgccttgagtaacagtgcccgtataaacagttaatgcaccctgcctatttcggaacctattattctgaaac
atgaaagtattaagaggctgagactcctcaagagaaggattaggattagcggggttttgctcagtaccaggcggataagtgc
cgtcgagagggttgatataagtatagcccggaataggtgtatcaccgtactcaggaggtttagtaccgccaccctcagaacc
gccaccctcagaaccgccaccctcagagccaccaccctcattttcagggatagcaagcccaataggaacccatgtaccgta
acactgagtttcgtcaccagtacaaaccacaacgcctgtagcattccacagacagccctcatagttagcgtaacgatctaaag
ttttgtcgtctttccagacgttagtaaatgaattttctgtatgaggttttgctaaacaactttcaacagtttcagcggagtgagaata
gaaaggaacaactaaaggaattgcgaataataattttttcacgttgaaaatctccaaaaaaaaaggctccaaaaggagccttt
aattgtatcggtttatcagcttgctttcgaggtgaatttcttaaacagcttgataccgatagttgcgccgacaatgacaacaacca
tcgcccacgcataaccgatatattcggtcgctgaggcttgcagggagtcaaaggccgcttttgcgggatcgtcaccctcagc
agcgaaagacagcatcggaacgagggtagcaacggctacggaggctttgaggactaaagactttttcatgaggaagtttcc
attaaacgggtaaaatacgtaatgccactacgaaggcaccaacctaaaacgaaagaggcgaaagaatacactaaaacact
catctttgacccccagcgattataccaagcgcgaaacaaagtacaacggagatttgtatcatcgcctgataaattgtgtcgaa
atccgcgacctgctccatgttacttagccggaacgaggcgcagacggtcaatcataagagaaccgaactgaccaactttga
acgaggacagatgcacggtgtacagaccaggcgcatacgctggctgaccttcgtcgagagtaatcttgacaagcaccgga
tattcattacccaaatcaacgtaacaaagctgctcattcagtgaataaggcttgccctgacgagaaacaccagaacgggtagt
aaattgcgcttgagacggtttaatttcaactttaatcattttgaattaccttatgcgattttaagaactggctcattataccagtcag
gacgttgggaggaaaaatctacgttaataaaacgaactaacggaacaacattattacaggtggaaagattcatcaattgagat
ttaggaataccacactcaactaatgcagatacataacgccaaaaggaattacgaggcatggtaagagcaacactatcataac
cctcattaaccagacgacgatagaaaccaaaatagcgagaggcttttgcaaaggaagttttgccagagggggtaattgtaaa
atgtttagactggatagcgtccaatactgcggaatcgtcataaatattcattgaatccccctcaaatgctttaaacagttcagaaa
acgagaatgaccataaatcaaaaatcaggtctttaccctgtctattatagtcagaagcaaagcgaattgcatcaaaaagattaa
gaggaagcccgaaagacttcaaatatcgcgtttcaattcgagcctcaaagcgaaccagaccggaagcaaattccaacaggt
caggattagacagtacctttaattgctccttttgataagaggtcatttttgcggatggcttagagcttaattgctgaatctggtgct
gtagttcaacatgttttaaatatgcaactaaagtacggtgtctggaagtttcattccatgtaacagttgattcccaattctgcgaac
gagtagatttagtttgaccattagatacatttcgcaaatggtcaataacctgtttagctatattttcatttggggcgcgagctgaaa
aggtggcatcaattctactaatggtagtagcgttaacatccaataaatcgtacaagcaaggcagagagttagcaaaattaagc
aataaagcctcagagcataaagctaaatcggttgtaccaaaaacattatgaccctgtaatacttttgctggtgaagccttaatttc
aacgcagggataaaaatttttagaaccctcatatattttaaatgcaatgccggagtaatgagtaggcaaagattcaaacgggt
gagaaaggccggagacagtcaaatcaccgtcaatatgatattcaaccgttctagctgataaattcatgccggagagggtagc
tatttttgagaggtctacaaaggctatcaggtcattacctgaaagtctggagcaaacaagagaatcgatgaacggtaatcgta
aaactagcatgtcaatcatatgtaccccggttgataatcagaaaagccccaaaaacaggatgattgtataagcaaatatttaaa
ttgtaaacgttaatgttttgttaaatttcgcgttaaatatttgttaaatcagcttattttttaaccagtaagcagaaaatgacaaaa
atccttataaatcaaaagaataggccgagttagttgtgagtgttgttccagtttggaacaagagtccactatt. (SEQ ID NO.
9).
TetA promoter region within SEQ ID NO. 9 removed during
cloning:
Cgattgcgtctcaacccctacttcggtatctgtattatcacgtgtatttttggtttcacggaaccaaaacat
aaccacaaggaaagtgacaatatttagcaacgcagcgataaaaaagggactatgcggtgaaatctctcctgcaaaaccacc
aataataggccccgctattaaaccaagcccaaaacttgcccctaaccaaccgaaccacttcacgcgttgagaagctgaggt
ggtatcggcaatgaccgatgccgcgacagccccagtagctcctgtgatccctgaaagcaaacggcctaaatacagcatcc
aaagcgcacttgaaaaagccagcaataagtaatccagcgatgcgcctattaatgacaacaacagcactgggcgccgacca
aatcggtcagacatttttccaagccaaggagcaaagataacctgcattaacgcataaagtgcaagcaatacgccaaagtggt
tagcgatatcttccgaagcaataaattcacgtaataacgttggcaagactggcatgataaggccaatccccatggcatcgagt
aacgtaattaccaatgcgatctttgtcgaactattcatttcacttttctctatcactgatagggagtggtaaaataactctatcaa
tgatagagtgtcaacaaaaattaggaattaatgatgtctaga. (SEQ ID NO. 10)
Example modified vector: fd-DZNR-DDRC, the capitalized portion
was inserted to replace the TetA region of the original vector sequence and
having the layout of: BioBrick Prefix-Promoter-BioBrick Scar-Ribosome
binding site-DZNR sequence-Ribosome binding site-DDRC sequence-
Transcriptional terminator-BioBrick Suffix
aaagaacgtggactccaacgtcaaagggcgaaaaaccgtctatcagggcgatggcccactacgtga
accatcacccaaatcaagtttttggaggtcgaggtgccgtaaagcactaaatcggaaccctaaagggatctcgggaaaagc
gttggtgaccaaaggtgccttttatcatcactttaaaaaaaaaaacaattactcagtgcctgttataagcagcaattaattatgat
tgatgcctacatcacaacaaaaactgatttaacaaatggttggtctgccttagaaagtatatttgaacattatcttgattatatta
ttgataataataaaaaccttatccctatccaagaagtgatgcctatcattggttggaatgaacttgaaaaaattagccttgaatac
attactggtaaggtaaacgccattgtcagcaaattgatccaagagaaccaacttaaagcttatgatagtgatgtgcttaaaaactta
ctcaatggctggtttatgcatatcgcaatacatgcgaaaaacctaaaagagcttgccgataaaaaaggccaatttattgctattt
accgcggctttttattgagcttgaaagataaataaaatagataggttttatttgaagctaaatcttctttatcgtaaaaaatgccct
cttgggttatcaagagggtcattatatttcgcggaataacatcatttggtgacgaaataactaagcacttgtctcctgtttactccc
ctgagcttgaggggttaacatgaaggtcatcgatagcaggataataatacagtaaaacgctaaaccaataatccaaatccagc
catcccaaattggtagtgaatgattataaataacagtaaacagtaatgggccaataacaccggttgcattggtaaggctcacc
aataatccctgtaaagcaccttgctcatgactctttgtttggatagacatcactccctgtaatgcaggtaaagcgatcccaccac
cagccaataaaattaaaacagggaaatctaaccaaccttcagatataaacgctaaaaaggcaaatgcactactatctgcaata
aattcgagcagtactgccgttttttcgccccatttagtggctattcttcctgccacaaaggcttggaatactgagtgtaaaagac
caagacccgctaatgaaaagccaaccatcatgctattccatccaaaacgattttcggtaaatagcacccacaccgttgcggg
aatttggcctatcaattgcgctgaaaaataaataatcaacaaaatgggcatcgttttaaataaagtgatgtataccgaattcGC
GGCCGCTTCTAGAGTTTACGGCTAGCTCAGTCCTAGGTACAATGCTAGCTA
CTAGAGAAGAAGGAGATATACCATGAAGAATAAATTTTATCCCAAAACCT
TTGAACGTGGTTACATCGGGAATTTGGAAGTAGAAAACCGCGCGATCCGC
ATGCCTATGGGAACAGAACTTGGCAACCCCGACGGTTCGCCCTCATGGGC
TTCCCTTAAGGCGTATGCTGAGGCTGCGGATGGTGGGACAGGTATTGTCTT
TATGGATAACGCAGGTGTTACTCAGTTCCACCATGTCGGTTTGTCGCTGGC
GTCAGATAATTATATCGGTCCTATGAGTGTCTTGGCGAAAACTATTAAGCA
GCATGGGGCAATTCCAGGTCTTCAAATTGTGCACCCTGGACGTGATGCTGC
ATTTGTGCGTGGCGACGATCTTATCAGTTCTAGCCGCATTCAGTGGGAGCC
TTGGTATGAAAATGGAGGGGCTGTTCCTCGCGAGCTGACAATCGAGGAAA
TTCATGATTTTGTGGGGTACTTCGGTGATTGTGCTTTACGTGCGCAAACAG
CGGGGTTCGAAATCGTTGATGTCCACGCGGCGTGCGGGGTCCTGTTGTCCA
ACTTCTTGTCACCTCGCAACAATACTCGTAACGATATGTATGGCGGCTCGT
TGCACAATCGTGCACGCTTTTTGTTAGAGGTAATCCGCGATATCAAAAAGA
AGTGCCCGAACCTTCCATTAGCTATTCGTCTTAGTGGAATCGACTTCGAGC
CCGATGGAATCACTATCGAAGAGACATGTGAGGTCGCGAAAATGTGTGAA
GCAGCTGGGGCTGACGCCATCAACATTACCTGGGGCTCTCATGCTGAGGT
AATCAATGCAGCAGGTTTGCTTTCTAAACATGGAGCGAACCACGTCGAGG
CCGCGAAAATGATCAAAGACGCGGTAAGCATCCCGACTATGTTATGTGGG
GGTATCTACAGCCCAGAAATTGGCGAAAAGTTACTTGAGGATGGTGTCTG
CGACTTCATCGGGATTGGTAAGCCAGCTCTGGCTGACCCCATGTGGGCTAA
AAAGGCGGCTGAGGGACGCCCCGAGGACATTCGTCCGTGCATCGGTTGTG
GAGTTGGATGCCACGATCGCGGGATGCTTTCCGGTGGAGTGGTACAATGC
GCAGTCAACGCTGCCCTGTACAAGTTTGATGAACCTGTCTACCCTCAAGCG
GAAGTTCCCAAGAAAGTTATTATTATTGGAGCGGGACCGGCAGGTTGTGA
AGCAGCCATTACAGCGAAAAAATGCGGGCACGACGTCACAATTTACGAGA
AGCGTAAAATCGGTGGCGTGCTGAAGGAGGCCACTGTGAGCGACTCCAAG
GAGGATCTGGGTCGCTTAATCACATATTACGAGACCCAGTTGAAAAAGGA
GGGAATCGAGGTAATCTATGAAGAAGCGACCGCGGATACCGTAGTGGCGG
GCGGTTTCGACGTTGCGATTGTTGCGTGTGGAGCCACAGTTCGCAATCTTA
ACATTGACGGCCAAGACGACCCAAGCGTTGTGTACGCTATGGACTTCCTG
GATAACGATTGCAAGTCAGACGCCGACCGTGTAGTAGTTGTCGGGGGCGG
AATTGTTGGCGCGGAAACTGCGCTTATTCTTGCGGAGGAGCGTGGGAAGG
ACGTAACCATTACAACTCGCTCACCAGAGTTCTTTGTATCTGGAGTGATGG
GGATTGCCTATATGGTACGTTTAGGCATGGCCGGTGTAACCATTAAGCCCA
GTACTCAATTGGTCGCCGTTAAGGATGGAAAGCCGATGTTCGCCGGGCCC
CGTGGTTTGGAAACTCTTGATGTGGACCAGACTATCATCTCTAGTGGCTTC
GTGCCCACGTTTAACCAATTCCGTGCACAGATTGAAGAAAAGTGCGAAGA
CGTGCGTGTGATCGGGATCGGCGATTGCAAAGCATCTCGTATGGTAATGG
ATGCCGTTCATGAGGGGTACATCGCAGGCTGTAACCTGTAATAGAAGAAG
GAGATATACCATGATTAAAGCACAATTAAATCGCATTGCGTTACGCGCTGC
CGATGCTGATAAAGCCGTCGAAGACCTTAACAAATTGTTAGGTGTAACCTT
CTACGGGCCGTACGATGACGAACACATGGGCCTGCGCGTTGCGTTACCGA
AATCGGGGGGCATTGAAGTCATGGCCCCGATGCACGATCACGACGCCATC
GGCGCATATAAGGCATTACAGACGGTCGGAGAGGGTATCAGTGGTATCGC
AATGCGCGTGGACGACTTTGATGCCGCCAAGGAACACTTCGCAAGTCTTG
GCTTAACGCCGGTTGTAGAGTTCAATCACGGAAAGTTTCGTGAGATGATCT
TTCCCCCTCAGCCGGCAACGCACAACCTGGAAATCGCTGTGAACGAATTTC
CAGATGCTAACGGCGCGGCCATTCAGGTGGCATTAGATATGGGTGTAGAC
TGGACTGATGTTTGTGACTGGGATGCGAAGTAGTAACTAGCATAACCCCTT
GGGGCCTCTAAACGGGTCTTGAGGGGTTTTTTGTACTAGattagataaaagtaaagtga
ttaacagcgcattagagctgcttaatgaggtcggaatcgaaggtttaacaacccgtaaactcgcccagaagctaggtgtaga
gcagcctacattgtattggcatgtaaaaaataagcgggctttgctcgacgccttagccattgagatgttagataggcaccatac
tcacttttgccctttagaaggggaaagctggcaagattttttacgtaataacgctaaaagttttagatgtgctttactaagtcatcg
cgatggagcaaaagtacatttaggtacacggcctacagaaaaacagtatgaaactctcgaaaatcaattagcctttttatgcca
acaaggtttttcactagagaatgcattatatgcactcagcgctgtggggcattttactttaggttgcgtattggaagatcaagag
catcaagtcgctaaagaagaaagggaaacacctactactgatagtatgccgccattattacgacaagctatcgaattatttgat
caccaaggtgcagagccagccttcttattcggccttgaattgatcatatgcggattagaaaaacaacttaaatgtgaaagtgg
gtcttaaaagcagcataacctttttccgtgatggtaacttcacggtaaccaagatgtcgagttaaccaccctttagattcataaa
gcgaaaataatgcggctccaacgtacccacctaaatggaaacggcgttcactccaatctaaacacgcacaacagattttacg
tgaatgtttggaaggaacgtcaattcccatttcatgaaaatattgaataccacttaatgtgatcattgaaccattttcagtgatcca
ttgctgttgacaaagggaatcatagatcccccgatttagagcttgacggggaaagccggagaacgtggcgagaaaggaag
ggaagaaagcgaaaggagcgggcgctagggcgctggcaagtgtagcggtcacgctgcgcgtaaccaccacacccgcc
gcgcttaatgcgccgctacagggcgcgtactatggttgctttgacgagcacgtacaacgtgctttcctcgttagaatcagaac
gggagctaaacaggaggccgattaaagggattttagacaggaacggcacaccagaatcttgagaagtgtttttgtaatcagt
gaggccaccgagcaaaagagtctgaccatcacgcaaattaaccgttgtcgcaatacttctttgattagtaataacatcacttgc
ctgagtagaagaactcaaactatcggccttactggttatatctaaaacaatattaccgccagccattgcaacgggaaaaacac
tcatagaaataccaacattttgacgctcaaccgtctgaaatggattatttacattggcagattcaccagttacacgaccagtaat
aaaagggacattctggccaacagaaatagaacccttctgacctgaaagcgtaagaatacgaggcacagacaatatttttgaa
tggctattagtctttaatgcgcgaactgatagccctaaaacatcgccgttaaaaataccgaacgaaccacccgcagaagata
aaacagaggttagacggtcagtattaacaccaccagcaacagtgccgcgctgagagccagcagcaaaggaaaaatctaa
agcatcaccttgctgaacctcgaaaattaatccttcaatcaatatctggtcagttggcaaatcaacagtagaaagaaattgcgg
aaggttatctaaaatatctttaggggcgctaactactaataagttagaaccatcaacagataatacatttgaggatttagatgtatt
agatttaacaaacaattctacaacccttattaaatcctttgcgcgaacgttattaattttaaatgtttgagtaacattatcattttg
cggaacaaagaaaccaccagaaggagcggaattatcatcatattcctgaatatcagatgatggcaattcatcaatataatcctga
ctatttgggttatatttctgaattatggaaggaattgagccaaccatatcaaaattattagcacgtaaaacagagataaagaaatt
gcgtaaattttcaggcttaacgtcagaggaatatacagtcactgtacctttaacatcaggtgagacaataacagattcacctgtt
tgctttgaataccaagtcacgaaatcgcgcagaggcgaattattcatttcaattacttgagcaaaagaagatgatgaaacaaa
catcaagaaaacaaaattaattacatttaacaatttcatttgaattaccttttttaattgaaacagtacataaatcaatatatgtga
tggaataaccttgcttctgtaaatcgtcgctattaattaattttcccttagaatccttgaaaacatagcgatagcttagattaagacgc
tgagaagagtcaatagtgaatttatcaaaatcataggtctgagagactacctttttaacctccggcttaggttgggttatataacta
tatgtaaatgctgatgcaaatcctatcgcaagacaaagaacgcgagaaaactttttcaaatatattttagttaatttcatcttctga
cctaaatttaatggtttgaaataccgaccgtgtgataaataaggggttaaatatgaataaacacctgaatcataattactggaaa
aagcctgtttagtgtcatatgcgttatataaattcttaccagtataaagccaacgctcaacagtagggcttaattgagaatcacc
atatttaacaacaccaacatgtaatttaggcagaggcatttttgagccagtaacaagagaatataaagtgccgacaaagggta
aagtaattctgtccagacggcgacaataaacaacgtgttcagctaatgcagaacgcgcctgtttatcaacaatagataaatcct
gaacaagaaaaataatatcccatcccaatttacgagcatgaagaaaccaatcaataatcggctgtctttccttgtcattccatga
acgggtattaaaccaagtaccgcattcatcaagaacaagcaaaccgtttttattttcgtcgtaggaatcattaccacgaccaata
gcaagcaaatcagaaatagaaggcttatccggtattctaagaacgcgaggcgttttagcgaacctcccgacttgcgggagg
ttttgaagccttaaatcaagattagttgctattttgcacccagctacaattttatcctgaatcttaccaacgctaacgagcgtctttc
cagagcctaatttgccagttacaaaataaacagccatatttatttatcccaatccaaataagaaacgattttttgtttaacgtcaaa
aatgaaaatagcagcctttacagagagaataacataaaaacagggaagcgcattagacgggagaattaactgaacgccct
gaacaaaatcagagggtaattgtgcgctaatatcagagagataacccacaagaattgagttaagcccaataataagagcaa
gaaacaatgaaatagcaatagctatcttaccgaagccctttttaaggaaagtaagcagatagccgaacaaagttaccagaag
gaaaccgaggaaacgcaataataacggaatacccaaaagaactggcatgattaagactccttattacgcagtatgttagcaa
acgtcgaaaatacatacataaaggtggcaacatataaaagaaacgcaaagacaccacggaataagtttattttgtcacaatca
atagaaaattcatatggtttaccagcgccaaagacataagggcgacattcaaccgactgaggcaaagaaggtaaatattgac
ggaaattattcattaaaggtgaattatcaccgtcaccgacttgagccatttgggaattagagccagcaaaatcaccagtagca
ccattaccattagcaaggccggaaacgtcaccaatgaaaccatcgatagcagcaccgtaatcagtagcgacagaatcaagt
ttgcctttagcgtcagactgtagcgcgttttcatcggcattttcggtcatagcccccttattagcgtttgccattttttcataatcaaa
atcaccggaaccggagccgccaccggaaccgccaccctcagagccgccaccctcagaaccgccaccctcagagccgc
caccctcagagccgccaccagaaccaccaccagagccgccgccagcattgacaggaggttgaggcaggtcagacgatt
ggccttgatattcacaaacgaatggatcctcattaaagccagaatggaaagcgcagtctctgaatttaccgttccagtaagcgt
catacatggcttttgatgatacaggagtgtactggtaataagttttaacggggtcagtgccttgagtaacagtgcccgtataaac
agttaatgcaccctgcctatttcggaacctattattctgaaacatgaaagtattaagaggctgagactcctcaagagaaggatt
aggattagcggggttttgctcagtaccaggcggataagtgccgtcgagagggttgatataagtatagcccggaataggtgta
tcaccgtactcaggaggtttagtaccgccaccctcagaaccgccaccctcagaaccgccaccctcagagccaccaccctc
attttcagggatagcaagcccaataggaacccatgtaccgtaacactgagtttcgtcaccagtacaaaccacaacgcctgta
gcattccacagacagccctcatagttagcgtaacgatctaaagttttgtcgtctttccagacgttagtaaatgaattttctgtatga
ggttttgctaaacaactttcaacagtttcagcggagtgagaatagaaaggaacaactaaaggaattgcgaataataattttttca
cgttgaaaatctccaaaaaaaaaggctccaaaaggagcctttaattgtatcggtttatcagcttgctttcgaggtgaatttcttaa
acagcttgataccgatagttgcgccgacaatgacaacaaccatcgcccacgcataaccgatatattcggtcgctgaggcttg
cagggagtcaaaggccgcttttgcgggatcgtcaccctcagcagcgaaagacagcatcggaacgagggtagcaacggct
acggaggctttgaggactaaagactttttcatgaggaagtttccattaaacgggtaaaatacgtaatgccactacgaaggcac
caacctaaaacgaaagaggcgaaagaatacactaaaacactcatctttgacccccagcgattataccaagcgcgaaacaaa
gtacaacggagatttgtatcatcgcctgataaattgtgtcgaaatccgcgacctgctccatgttacttagccggaacgaggcg
cagacggtcaatcataagagaaccgaactgaccaactttgaacgaggacagatgcacggtgtacagaccaggcgcatac
gctggctgaccttcgtcgagagtaatcttgacaagcaccggatattcattacccaaatcaacgtaacaaagctgctcattcagt
gaataaggcttgccctgacgagaaacaccagaacgggtagtaaattgcgcttgagacggtttaatttcaactttaatcattttga
attaccttatgcgattttaagaactggctcattataccagtcaggacgttgggaggaaaaatctacgttaataaaacgaactaac
ggaacaacattattacaggtggaaagattcatcaattgagatttaggaataccacactcaactaatgcagatacataacgcca
aaaggaattacgaggcatggtaagagcaacactatcataaccctcattaaccagacgacgatagaaaccaaaatagcgag
aggcttttgcaaaggaagttttgccagagggggtaattgtaaaatgtttagactggatagcgtccaatactgcggaatcgtcat
aaatattcattgaatccccctcaaatgctttaaacagttcagaaaacgagaatgaccataaatcaaaaatcaggtctttaccctg
tctattatagtcagaagcaaagcgaattgcatcaaaaagattaagaggaagcccgaaagacttcaaatatcgcgtttcaattc
gagcctcaaagcgaaccagaccggaagcaaattccaacaggtcaggattagacagtacctttaattgctccttttgataaga
ggtcatttttgcggatggcttagagcttaattgctgaatctggtgctgtagttcaacatgttttaaatatgcaactaaagtacggtg
tctggaagtttcattccatgtaacagttgattcccaattctgcgaacgagtagatttagtttgaccattagatacatttcgcaaatg
gtcaataacctgtttagctatattttcatttggggcgcgagctgaaaaggtggcatcaattctactaatggtagtagcgttaacat
ccaataaatcgtacaagcaaggcagagagttagcaaaattaagcaataaagcctcagagcataaagctaaatcggttgtacc
aaaaacattatgaccctgtaatacttttgctggtgaagccttaatttcaacgcagggataaaaatttttagaaccctcatatatttta
aatgcaatgccggagtaatgagtaggcaaagattcaaacgggtgagaaaggccggagacagtcaaatcaccgtcaatatg
atattcaaccgttctagctgataaattcatgccggagagggtagctatttttgagaggtctacaaaggctatcaggtcattacct
gaaagtctggagcaaacaagagaatcgatgaacggtaatcgtaaaactagcatgtcaatcatatgtaccccggttgataatc
agaaaagccccaaaaacaggatgattgtataagcaaatatttaaattgtaaacgttaatgttttgttaaatttcgcgttaaatattt
gttaaatcagcttattttttaaccagtaagcagaaaatgacaaaaatccttataaatcaaaagaataggccgagttagttgtgag
tgttgttccagtttggaacaagagtccactatt. (SEQ ID NO. 11).

1-24. (canceled)

25. A method for modifying the phenotype of a mammalian gut comprising:
introducing to the mammalian gut a probiotic for the treatment of cardiovascular disease (CVD) comprising:
a bacterium from the genus Escherichia containing therein at least two stable plasmids for isoflavone metabolism, said plasmids providing one or more components of isoflavone metabolism for converting Daidzein to Equol, Genistein to 5-Hydroxy-Equol, or both.

26. The method of claim 25, further comprising co-administering a transduction agent.

27. The method of claim 26, wherein the transduction agent comprises an antibiotic, a polycation, a bacteriophage, or a combination of two or more of the foregoing.

28. The method of claim 25, wherein introducing the probiotic to the mammalian gut is performed according to a dosage regimen.

29. The method of claim 28, wherein the dosage regimen comprises:
introducing the probiotic having a concentration of the bacterium to the mammalian gut at a dosing schedule over the course of a time period.

30. The method of claim 29, wherein the concentration is between 1×10⁶ to 1×10¹⁰ CFU/mL.

31. The method of claim 29, wherein the time period is selected from: every 4 hours, every 6 hours, every 12 hours, every 24 hours, or every 48 hours.

32. The method of claim 29, wherein the time period is 1 day, 1 week, 1 month, or one year.

33. The method of claim 25, wherein the probiotic comprises:
a first bacterium having a first plasmid comprising the nucleotide sequence or sequences encoding DZNR, DDRC, or both; and
a second bacterium having a second plasmid comprising the nucleotide sequence or sequences encoding DHDR, THDR, or both.

34. The method of claim 33, wherein the ratio of the first bacterium to the second bacterium is no less than 9:1 and no greater than 1:9.

35-37. (canceled)

38. The method of claim 33, wherein the nucleotide sequence encoding Daidzein reductase (DZNR) comprises the sequence of SEQ ID 1.

39. The method of claim 33, wherein the nucleotide sequence encoding Dihydrodaidzein racemase (DDRC) comprises the sequence of SEQ ID 2.

40. The method of claim 33, wherein the nucleotide sequence encoding Dihydrodaidzein reductase (DHDR) comprises the sequence of SEQ ID 3.

41. The method of claim 33, wherein nucleotide sequence encoding Tetrahydrodaidzein reductase (THDR) comprises the sequence of SEQ ID 4.

42. The method of claim 25, wherein the first plasmid further comprises an antibiotic resistance gene, and wherein the second plasmid further comprises an antibiotic resistance gene.

43. The method of claim 42, wherein the antibiotic resistance gene of the first plasmid or the antibiotic resistance gene of the second plasmid is an ampicillin or a kanamycin resistance gene.

44. The method of claim 25, wherein the probiotic comprises a dosage form that can be taken orally.

45. The method of claim 44, wherein the dosage form comprises a liquid, a foodstuff, or a capsule.


SEQUENCE LISTING (11 sequences)

SEQ ID NO: 1 - DNA, 1938 bp, Lactococcus garvieae
  source 1..1938: mol_type = genomic DNA
atgaagaataaattttatcccaaaacctttgaacgtggttacatcgggaatttggaagtagaaaaccgcgcgatccgcatgcctatgggaacagaacttggcaaccccgacggttcgccctcatgggcttcccttaaggcgtatgctgaggctgcggatggtgggacaggtattgtctttatggataacgcaggtgttactcagttccaccatgtcggtttgtcgctggcgtcagataattatatcggtcctatgagtgtcttggcgaaaactattaagcagcatggggcaattccaggtcttcaaattgtgcaccctggacgtgatgctgcatttgtgcgtggcgacgatcttatcagttctagccgcattcagtgggagccttggtatgaaaatggaggggctgttcctcgcgagctgacaatcgaggaaattcatgattttgtggggtacttcggtgattgtgctttacgtgcgcaaacagcggggttcgaaatcgttgatgtccacgcggcgtgcggggtcctgttgtccaacttcttgtcacctcgcaacaatactcgtaacgatatgtatggcggctcgttgcacaatcgtgcacgctttttgttagaggtaatccgcgatatcaaaaagaagtgcccgaaccttccattagctattcgtcttagtggaatcgacttcgagcccgatggaatcactatcgaagagacatgtgaggtcgcgaaaatgtgtgaagcagctggggctgacgccatcaacattacctggggctctcatgctgaggtaatcaatgcagcaggtttgctttctaaacatggagcgaaccacgtcgaggccgcgaaaatgatcaaagacgcggtaagcatcccgactatgttatgtgggggtatctacagcccagaaattggcgaaaagttacttgaggatggtgtctgcgacttcatcgggattggtaagccagctctggctgaccccatgtgggctaaaaaggcggctgagggacgccccgaggacattcgtccgtgcatcggttgtggagttggatgccacgatcgcgggatgctttccggtggagtggtacaatgcgcagtcaacgctgccctgtacaagtttgatgaacctgtctaccctcaagcggaagttcccaagaaagttattattattggagcgggaccggcaggttgtgaagcagccattacagcgaaaaaatgcgggcacgacgtcacaatttacgagaagcgtaaaatcggtggcgtgctgaaggaggccactgtgagcgactccaaggaggatctgggtcgcttaatcacatattacgagacccagttgaaaaaggagggaatcgaggtaatctatgaagaagcgaccgcggataccgtagtggcgggcggtttcgacgttgcgattgttgcgtgtggagccacagttcgcaatcttaacattgacggccaagacgacccaagcgttgtgtacgctatggacttcctggataacgattgcaagtcagacgccgaccgtgtagtagttgtcgggggcggaattgttggcgcggaaactgcgcttattcttgcggaggagcgtgggaaggacgtaaccattacaactcgctcaccagagttctttgtatctggagtgatggggattgcctatatggtacgtttaggcatggccggtgtaaccattaagcccagtactcaattggtcgccgttaaggatggaaagccgatgttcgccgggccccgtggtttggaaactcttgatgtggaccagactatcatctctagtggcttcgtgcccacgtttaaccaattccgtgcacagattgaagaaaagtgcgaagacgtgcgtgtgatcgggatcggcgattgcaaagcatctcgtatggtaatggatgccgttcatgaggggtacatcgcaggctgtaacctgtaatag

SEQ ID NO: 2 - DNA, 480 bp, Lactococcus garvieae
  source 1..480: mol_type = genomic DNA
atgattaaagcacaattaaatcgcattgcgttacgcgctgccgatgctgataaagccgtcgaagaccttaacaaattgttaggtgtaaccttctacgggccgtacgatgacgaacacatgggcctgcgcgttgcgttaccgaaatcggggggcattgaagtcatggccccgatgcacgatcacgacgccatcggcgcatataaggcattacagacggtcggagagggtatcagtggtatcgcaatgcgcgtggacgactttgatgccgccaaggaacacttcgcaagtcttggcttaacgccggttgtagagttcaatcacggaaagtttcgtgagatgatctttccccctcagccggcaacgcacaacctggaaatcgctgtgaacgaatttccagatgctaacggcgcggccattcaggtggcattagatatgggtgtagactggactgatgtttgtgactgggatgcgaagtagtaa

SEQ ID NO: 3 - DNA, 864 bp, Lactococcus garvieae
  source 1..864: mol_type = genomic DNA
atggctcaggaagtgaaagtaccaaaaatgcccggtgcacctgtttttggtaaatggatctcaccggaggagtccgtgggtcagcgcctgaagggtaagaagattttgttgaccgggactactaagggggtgggccgtgtaacgcaagagttactgtgtgcacatggagcgttcgtgtgtgggagtggccgcacgccaggggtggctgcatcagttgccgacgaattgaaagcaaagggttatcaggccgcaggaatggatgtagacctttctgactacgatgctgtaaaaaagtgggtcgaggagtgcgccgaactgatgggtggaattgatgttgtgatcaacaatgcctcacaccctggcatggcacccttcggtgagatgacaccggaaatctggaattacggaatcaagaacgagctggatttagtgtacaacgtatgcaattgtgcatggccgtatttacaaaaagccgatggggcatcaatcattattacgtcaagcaccgtagcgttacaagggagcaactcacctcaggcatgtcacgcagcttgtaaaggggcctgtctttcactggcacgtcagcttgccgcggaaggtgggccattcggaatccgctgtaactcggttacgcccggccttgtttggactgaggcaatgagtaacatcccgaaggaaatggcctctggtttggttgccgctcaaacaactcagcaggccgttgacccgatggacattgcttacgcctatctgttcttagcctctgacgaaagtcgccaaattactgccgcgaatatccctgtggatggaggttgtgcaggtgccgtaaccggtggaatgcagggcgaaatcgaagtataatag

SEQ ID NO: 4 - DNA, 1464 bp, Lactococcus garvieae
  source 1..1464: mol_type = genomic DNA
atggcggagtttgacgtagagtatgatttggtagtcgtcggcgggggggcttccggcaaatcagctgccttgattgctgcccgcgaagggaaacgtgttgtcgttttggagaagatgccggagacggggggtttgtcgatgtacgccgagggcacagcagcctttgagtctagcattcagaacgaattgggaactccccgccttagtaaatatcactttcccacgaaacaggaaggcatcgaaaagtttatggggtattcgcatcaacgtgctaattacgacgtagttcgtgccttcgtcgaaaatagcgccgaaaccatcgacatctaccgtgacctgggtgtagtatataaagcatgcgacatcgccgctgaagacgatccaaacgaggtctggacgtttcacttgcctgagggtcttggagcacattgtcaggaagttctgcttgacgcgattcaaaaactggatgttgatattttcacatccactccggcgaaggaacttatcattgaagacggggctgtcgtaggagtcgtcgctgaatccgacggcgagcctctgcgtgtgggtggcaaagctgtcatccttgcgaccggaggtatgggatcaagtccagaacgtatcttcaaatattcttggtttgcgcccgcggcatataatatgaacactttaacaccgcttcagaacgtaggggatggcttagaccttgcactgtcagccggggccgacccgacctacatcactacttgtccaatcctggcagcaggaggccgcgacatgactatggacagccaggtgggaggtgccggtgttaatccgggggtgtggattaacaaaacgggacgccgttttgcggccgaatcagtagcggagaatatcggggacattggaacatattatgggaaacagccgggtggagttgtgtggagcattttgtctcaggcggacatcgatcgcttggtcgccgaaggctctgagattgccattggagagtttgttgtttaccataagcctatggaacgcctgcccatcgagctggaggcccacctggagagcgggttagttaaaaaggcgggaagttttgaagaacttgccgctttaattgacgtacccgtcgacactttcgttgcaacaatggcagattataacgaggcgtgtgagaaagggtatgatgatgcttttatgaagaaaccccagtacttgcgccccatggttgaaggtccgttctatgctatcccacttgcaaccggaaccatgggatcggccggaggtattcgcatcaacggaaatatgcaagtcgtagacgcagattataatgcgattccaggtctttatgccgtagggttggacgcgaccggtttgtacggtgacagttacaatatggaagttccgggggccgccaatgggttcgctcatacgtcaggacgtattgcagctcgtcacgctatctccacgatgtagtaa

SEQ ID NO: 5 - DNA, 35 bp, synthetic construct
  source 1..35: mol_type = other DNA
tttacggctagctcagtcctaggtacaatgctagc

SEQ ID NO: 6 - skipped

SEQ ID NO: 7 - DNA, 16 bp, synthetic construct
  source 1..16: mol_type = other DNA
aagaaggagatatacc

SEQ ID NO: 8 - DNA, 48 bp, synthetic construct
  source 1..48: mol_type = other DNA
ctagcataaccccttggggcctctaaacgggtcttgaggggttttttg

SEQ ID NO: 9 - DNA, 9183 bp, synthetic construct
  source 1..9183: mol_type = other DNA
aaagaacgtggactccaacgtcaaagggcgaaaaaccgtctatcagggcgatggcccactacgtgaaccatcacccaaatcaagtttttggaggtcgaggtgccgtaaagcactaaatcggaaccctaaagggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaattagccttgaatacattactggtaaggtaaacgccattgtcagcaaattgatccaagagaaccaacttaaagcttatgatagtgatgtgcttaaaaacttactcaatggctggtttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagtaaacagtaatgggccaataacaccggttgcattggtaaggctcaccaataatccctgtaaagcaccttgctcatgactctttgtttggatagacatcactccctgtaatgcaggtaaagcgatcccaccaccagccaataaaattaaaacagggaaatctaaccaaccttcagatataaacgctaaaaaggcaaatgcactactatctgcaataaattcgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtataccgaattcgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtggtaaaataactctatcaatgatagagtgtcaacaaaaattaggaattaatgatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttaaccaccctttagattcataaagcgaaaataatgcggctccaacgtacccacctaaatggaaacggcgttcactccaatctaaacacgcacaacagattttacgtgaatgtttggaaggaacgtcaattcccatttcatgaaaatattgaataccacttaatgtgatcattgaaccattttcagtgatccattgctgttgacaaagggaatcatagatcccccgatttagagcttgacggggaaagccggagaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtacaacgtgctttcctcgttagaatcagaacgggagctaaacaggaggccgattaaagggattttagacaggaacggcacaccagaatcttgagaagtgtttttgtaatcagtgaggccaccgagcaaaagagtctgaccatcacgcaaattaaccgttgtcgcaatacttctttgattagtaataacatcacttgcctgagtagaagaactcaaactatcggccttactggttatatctaaaacaatattaccgccagccattgcaacgggaaaaacactcatagaaataccaacattttgacgctcaaccgtctgaaatggattatttacattggcagattcaccagttacacgaccagtaataaaagggacattctggccaacagaaatagaacccttctgacctgaaagcgtaagaatacgaggcacagacaatatttttgaatggctattagtctttaatgcgcgaactgatagccctaaaacatcgccgttaaaaataccgaacgaaccacccgcagaagataaaacagaggttagacggtcagtattaacaccaccagcaacagtgccgcgctgagagccagcagcaaaggaaaaatctaaagcatcaccttgctgaacctcgaaaattaatccttcaatcaatatctggtcagttggcaaatcaacagtagaaagaaattgcggaaggttatctaaaatatctttaggggcgctaactactaataagttagaaccatcaacagataatacatttgaggatttagatgtattagatttaacaaacaattctacaacccttattaaatcctttgcgcgaacgttattaattttaaatgtttgagtaacattatcattttgcggaacaaagaaaccaccagaaggagcggaattatcatcatattcctgaatatcagatgatggcaattcatcaatataatcctgactatttgggttatatttctgaattatggaaggaattgagccaaccatatcaaaattattagcacgtaaaacagagataaagaaattgcgtaaattttcaggcttaacgtcagaggaatatacagtcactgtacctttaacatcaggtgagacaataacagattcacctgtttgctttgaataccaagtcacgaaatcgcgcagaggcgaattattcatttcaattacttgagcaaaagaagatgatgaaacaaacatcaagaaaacaaaattaattacatttaacaatttcatttgaattaccttttttaattgaaacagtacataaatcaatatatgtgatggaataaccttgcttctgtaaatcgtcgctattaattaattttcccttagaatccttgaaaacatagcgatagcttagattaagacgctgagaagagtcaatagtgaatttatcaaaatcataggtctgagagactacctttttaacctccggcttaggttgggttatataactatatgtaaatgctgatgcaaatcctatcgcaagacaaagaacgcgagaaaactttttcaaatatattttagttaatttcatcttctgacctaaatttaatggtttgaaataccgaccgtgtgataaataaggggttaaatatgaataaacacctgaatcataattactggaaaaagcctgtttagtgtcatatgcgttatataaattcttaccagtataaagccaacgctcaacagtagggcttaattgagaatcaccatatttaacaacaccaacatgtaatttaggcagaggcatttttgagccagtaacaagagaatataaagtgccgacaaagggtaaagtaattctgtccagacggcgacaataaacaacgtgttcagctaatgcagaacgcgcctgtttatcaacaatagataaatcctgaacaagaaaaataatatcccatcccaatttacgagcatgaagaaaccaatcaataatcggctgtctttccttgtcattccatgaacgggtattaaaccaagtaccgcattcatcaagaacaagcaaaccgtttttattttcgtcgtaggaatcattaccacgaccaatagcaagcaaatcagaaatagaaggcttatccggtattctaagaacgcgaggcgttttagcgaacctcccgacttgcgggaggttttgaagccttaaatcaagattagttgctattttgcacccagctacaattttatcctgaatcttaccaacgctaacgagcgtctttccagagcctaatttgccagttacaaaataaacagccatatttatttatcccaatccaaataagaaacgattttttgtttaacgtcaaaaatgaaaatagcagcctttacagagagaataacataaaaacagggaagcgcattagacgggagaattaactgaacgccctgaacaaaatcagagggtaattgtgcgctaatatcagagagataacccacaagaattgagttaagcccaataataagagcaagaaacaatgaaatagcaatagctatcttaccgaagccctttttaaggaaagtaagcagatagccgaacaaagttaccagaaggaaaccgaggaaacgcaataataacggaatacccaaaagaactggcatgattaagactccttattacgcagtatgttagcaaacgtcgaaaatacatacataaaggtggcaacatataaaagaaacgcaaagacaccacggaataagtttattttgtcacaatcaatagaaaattcatatggtttaccagcgccaaagacataagggcgacattcaaccgactgaggcaaagaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccattttttcataatcaaaatcaccggaaccggagccgccaccggaaccgccaccctcagagccgccaccctcagaaccgccaccctcagagccgccaccctcagagccgccaccagaaccaccaccagagccgccgccagcattgacaggaggttgaggcaggtcagacgattggccttgatattcacaaacgaatggatcctcattaaagccagaatggaaagcgcagtctctgaatttaccgttccagtaagcgtcatacatggcttttgatgatacaggagtgtactggtaataagttttaacggggtcagtgccttgagtaacagtgcccgtataaacagttaatgcaccctgcctatttcggaacctattattctgaaacatgaaagtattaagaggctgagactcctcaagagaaggattaggattagcggggttttgctcagtaccaggcggataagtgccgtcgagagggttgatataagtatagcccggaataggtgtatcaccgtactcaggaggtttagtaccgccaccctcagaaccgccaccctcagaaccgccaccctcagagccaccaccctcattttcagggatagcaagcccaataggaacccatgtaccgtaacactgagtttcgtcaccagtacaaaccacaacgcctgtagcattccacagacagccctcatagttagcgtaacgatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcgggatcgtcaccctcagcagcgaaagacagcatcggaacgagggtagcaacggctacggaggctttgaggactaaagactttttcatgaggaagtttccattaaacgggtaaaatacgtaatgccactacgaaggcaccaacctaaaacgaaagaggcgaaagaatacactaaaacactcatctttgacccccagcgattataccaagcgcgaaacaaagtacaacggagatttgtatcatcgcctgataaattgtgtcgaaatccgcgacctgctccatgttacttagccggaacgaggcgcagacggtcaatcataagagaaccgaactgaccaactttgaacgaggacagatgcacggtgtacagaccaggcgcatacgctggctgaccttcgtcgagagtaatcttgacaagcaccggatattcattacccaaatcaacgtaacaaagctgctcattcagtgaataaggcttgccctgacgagaaacaccagaacgggtagtaaattgcgcttgagacggtttaatttcaactttaatcattttgaattaccttatgcgattttaagaactggctcattataccagtcaggacgttgggaggaaaaatctacgttaataaaacgaactaacggaacaacattattacaggtggaaagattcatcaattgagatttaggaataccacactcaactaatgcagatacataacgccaaaaggaattacgaggcatggtaagagcaacactatcataaccctcattaaccagacgacgatagaaaccaaaatagcgagaggcttttgcaaaggaagttttgccagagggggtaattgtaaaatgtttagactggatagcgtccaatactgcggaatcgtcataaatattcattgaatccccctcaaatgctttaaacagttcagaaaacgagaatgaccataaatcaaaaatcaggtctttaccctgtctattatagtcagaagcaaagcgaattgcatcaaaaagattaagaggaagcccgaaagacttcaaatatcgcgtttcaattcgagcctcaaagcgaaccagaccggaagcaaattccaacaggtcaggattagacagtacctttaattgctccttttgataagaggtcatttttgcggatggcttagagcttaattgctgaatctggtgctgtagttcaacatgttttaaatatgcaactaaagtacggtgtctggaagtttcattccatgtaacagttgattcccaattctgcgaacgagtagatttagtttgaccattagatacatttcgcaaatggtcaataacctgtttagctatattttcatttggggcgcgagctgaaaaggtggcatcaattctactaatggtagtagcgttaacatccaataaatcgtacaagcaaggcagagagttagcaaaattaagcaataaagcctcagagcataaagctaaatcggttgtaccaaaaacattatgaccctgtaatacttttgctggtgaagccttaatttcaacgcagggataaaaatttttagaaccctcatatattttaaatgcaatgccggagtaatgagtaggcaaagattcaaacgggtgagaaaggccggagacagtcaaatcaccgtcaatatgatattcaaccgttctagctgataaattcatgccggagagggtagctatttttgagaggtctacaaaggctatcaggtcattacctgaaagtctggagcaaacaagagaatcgatgaacggtaatcgtaaaactagcatgtcaatcatatgtaccccggttgataatcagaaaagccccaaaaacaggatgattgtataagcaaatatttaaattgtaaacgttaatgttttgttaaatttcgcgttaaatatttgttaaatcagcttattttttaaccagtaagcagaaaatgacaaaaatccttataaatcaaaagaataggccgagttagttgtgagtgttgttccagtttggaacaagagtccactatt

SEQ ID NO: 10 - DNA, 687 bp, synthetic construct
  source 1..687: mol_type = other DNA
cgattgcgtctcaacccctacttcggtatctgtattatcacgtgtatttttggtttcacggaaccaaaacataaccacaaggaaagtgacaatatttagcaacgcagcgataaaaaagggactatgcggtgaaatctctcctgcaaaaccaccaataataggccccgctattaaaccaagcccaaaacttgcccctaaccaaccgaaccacttcacgcgttgagaagctgaggtggtatcggcaatgaccgatgccgcgacagccccagtagctcctgtgatccctgaaagcaaacggcctaaatacagcatccaaagcgcacttgaaaaagccagcaataagtaatccagcgatgcgcctattaatgacaacaacagcactgggcgccgaccaaatcggtcagacatttttccaagccaaggagcaaagataacctgcattaacgcataaagtgcaagcaatacgccaaagtggttagcgatatcttccgaagcaataaattcacgtaataacgttggcaagactggcatgataaggccaatccccatggcatcgagtaacgtaattaccaatgcgatctttgtcgaactattcatttcacttttctctatcactgatagggagtggtaaaataactctatcaatgatagagtgtcaacaaaaattaggaattaatgatgtctaga

SEQ ID NO: 11 - DNA, 11061 bp, synthetic construct
  source 1..11061: mol_type = other DNA
aaagaacgtggactccaacgtcaaagggcgaaaaaccgtctatcagggcgatggcccactacgtgaaccatcacccaaatcaagtttttggaggtcgaggtgccgtaaagcactaaatcggaaccctaaagggatctcgggaaaagcgttggtgaccaaaggtgccttttatcatcactttaaaaataaaaaacaattactcagtgcctgttataagcagcaattaattatgattgatgcctacatcacaacaaaaactgatttaacaaatggttggtctgccttagaaagtatatttgaacattatcttgattatattattgataataataaaaaccttatccctatccaagaagtgatgcctatcattggttggaatgaacttgaaaaaattagccttgaatacattactggtaaggtaaacgccattgtcagcaaattgatccaagagaaccaacttaaagcttatgatagtgatgtgcttaaaaacttactcaatggctggtttatgcatatcgcaatacatgcgaaaaacctaaaagagcttgccgataaaaaaggccaatttattgctatttaccgcggctttttattgagcttgaaagataaataaaatagataggttttatttgaagctaaatcttctttatcgtaaaaaatgccctcttgggttatcaagagggtcattatatttcgcggaataacatcatttggtgacgaaataactaagcacttgtctcctgtttactcccctgagcttgaggggttaacatgaaggtcatcgatagcaggataataatacagtaaaacgctaaaccaataatccaaatccagccatcccaaattggtagtgaatgattataaataacagtaaacagtaatgggccaataacaccggttgcattggtaaggctcaccaataatccctgtaaagcaccttgctcatgactctttgtttggatagacatcactccctgtaatgcaggtaaagcgatcccaccaccagccaataaaattaaaacagggaaatctaaccaaccttcagatataaacgctaaaaaggcaaatgcactactatctgcaataaattcgagcagtactgccgttttttcgccccatttagtggctattcttcctgccacaaaggcttggaatactgagtgtaaaagaccaagacccgctaatgaaaagccaaccatcatgctattccatccaaaacgattttcggtaaatagcacccacaccgttgcgggaatttggcctatcaattgcgctgaaaaataaataatcaacaaaatgggcatcgttttaaataaagtgatgtataccgaattcgcggccgcttctagagtttacggctagctcagtcctaggtacaatgctagctactagagaagaaggagatataccatgaagaataaattttatcccaaaacctttgaacgtggttacatcgggaatttggaagtagaaaaccgcgcgatccgcatgcctatgggaacagaacttggcaaccccgacggttcgccctcatgggcttcccttaaggcgtatgctgaggctgcggatggtgggacaggtattgtctttatggataacgcaggtgttactcagttccaccatgtcggtttgtcgctggcgtcagataattatatcggtcctatgagtgtcttggcgaaaactattaagcagcatggggcaattccaggtcttcaaattgtgcaccctggacgtgatgctgcatttgtgcgtggcgacgatcttatcagttctagccgcattcagtgggagccttggtatgaaaatggaggggctgttcctcgcgagctgacaatcgaggaaattcatgattttgtggggtacttcggtgattgtgctttacgtgcgcaaacagcggggttcgaaatcgttgatgtccacgcggcgtgcggggtcctgttgtccaacttcttgtcacctcgcaacaatactcgtaacgatatgtatggcggctcgttgcacaatcgtgcacgctttttgttagaggtaatccgcgatatcaaaaagaagtgcccgaaccttccattagctattcgtcttagtggaatcgacttcgagcccgatggaatcactatcgaagagacatgtgaggtcgcgaaaatgtgtgaagcagctggggctgacgccatcaacattacctggggctctcatgctgaggtaatcaatgcagcaggtttgctttctaaacatggagcgaaccacgtcgaggccgcgaaaatgatcaaagacgcggtaagcatcccgactatgttatgtgggggtatctacagcccagaaattggcgaaaagttacttgaggatggtgtctgcgacttcatcgggattggtaagccagctctggctgaccccatgtgggctaaaaaggcggctgagggacgccccgaggacattcgtccgtgcatcggttgtggagttggatgccacgatcgcgggatgctttccggtggagtggtacaatgcgcagtcaacgctgccctgtacaagtttgatgaacctgtctaccctcaagcggaagttcccaagaaagttattattattggagcgggaccggcaggttgtgaagcagccattacagcgaaaaaatgcgggcacgacgtcacaatttacgagaagcgtaaaatcggtggcgtgctgaaggaggccactgtgagcgactccaaggaggatctgggtcgcttaatcacatattacgagacccagttgaaaaaggagggaatcgaggtaatctatgaagaagcgaccgcggataccgtagtggcgggcggtttcgacgttgcgattgttgcgtgtggagccacagttcgcaatcttaacattgacggccaagacgacccaagcgttgtgtacgctatggacttcctggataacgattgcaagtcagacgccgaccgtgtagtagttgtcgggggcggaattgttggcgcggaaactgcgcttattcttgcggaggagcgtgggaaggacgtaaccattacaactcgctcaccagagttctttgtatctggagtgatggggattgcctatatggtacgtttaggcatggccggtgtaaccattaagcccagtactcaattggtcgccgttaaggatggaaagccgatgttcgccgggccccgtggtttggaaactcttgatgtggaccagactatcatctctagtggcttcgtgcccacgtttaaccaattccgtgcacagattgaagaaaagtgcgaagacgtgcgtgtgatcgggatcggcgattgcaaagcatctcgtatggtaatggatgccgttcatgaggggtacatcgcaggctgtaacctgtaatagaagaaggagatataccatgattaaagcacaattaaatcgcattgcgttacgcgctgccgatgctgataaagccgtcgaagaccttaacaaattgttaggtgtaaccttctacgggccgtacgatgacgaacacatgggcctgcgcgttgcgttaccgaaatcggggggcattgaagtcatggccccgatgcacgatcacgacgccatcggcgcatataaggcattacagacggtcggagagggtatcagtggtatcgcaatgcgcgtggacgactttgatgccgccaaggaacacttcgcaagtcttggcttaacgccggttgtagagttcaatcacggaaagtttcgtgagatgatctttccccctcagccggcaacgcacaacctggaaatcgctgtgaacgaatttccagatgctaacggcgcggccattcaggtggcattagatatgggtgtagactggactgatgtttgtgactgggatgcgaagtagtaactagcataaccccttggggcctctaaacgggtcttgaggggttttttgtactagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtcttaaaagcagcataacctttttccgtgatggtaacttcacggtaaccaagatgtcgagttaaccaccctttagattcataaagcgaaaataatgcggctccaacgtacccacctaaatggaaacggcgttcactccaatctaaacacgcacaacagattttacgtgaatgtttggaaggaacgtcaattcccatttcatgaaaatattgaataccacttaatgtgatcattgaaccattttcagtgatccattgctgttgacaaagggaatcatagatcccccgatttagagcttgacggggaaagccggagaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtactatggttgctttgacgagcacgtacaacgtgctttcctcgttagaatcagaacgggagctaaacaggaggccgattaaagggattttagacaggaacggcacaccagaatcttgagaagtgtttttgtaatcagtgaggccaccgagcaaaagagtctgaccatcacgcaaattaaccgttgtcgcaatacttctttgattagtaataacatcacttgcctgagtagaagaactcaaactatcggccttactggttatatctaaaacaatattaccgccagccattgcaacgggaaaaacactcatagaaataccaacattttgacgctcaaccgtctgaaatggattatttacattggcagattcaccagttacacgaccagtaataaaagggacattctggccaacagaaatagaacccttctgacctgaaagcgtaagaatacgaggcacagacaatatttttgaatggctattagtctttaatgcgcgaactgatagccctaaaacatcgccgttaaaaataccgaacgaaccacccgcagaagataaaacagaggttagacggtcagtattaacaccaccagcaacagtgccgcgctgagagccagcagcaaaggaaaaatctaaagcatcaccttgctgaacctcgaaaattaatccttcaatcaatatctggtcagttggcaaatcaacagtagaaagaaattgcggaaggttatctaaaatatctttaggggcgctaactactaataagttagaaccatcaacagataatacatttgaggatttagatgtattagatttaacaaacaattctacaacccttattaaatcctttgcgcgaacgttattaattttaaatgtttgagtaacattatcattttgcggaacaaagaaaccaccagaaggagcggaattatcatcatattcctgaatatcagatgatggcaattcatcaatataatcctgactatttgggttatatttctgaattatggaaggaattgagccaaccatatcaaaattattagcacgtaaaacagagataaagaaattgcgtaaattttcaggcttaacgtcagaggaatatacagtcactgtacctttaacatcaggtgagacaataacagattcacctgtttgctttgaataccaagtcacgaaatcgcgcagaggcgaattattcatttcaattacttgagcaaaagaagatgatgaaacaaacatcaagaaaacaaaattaattacatttaacaatttcatttgaattaccttttttaattgaaacagtacataaatcaatatatgtgatggaataaccttgcttctgtaaatcgtcgctattaattaattttcccttagaatccttgaaaacatagcgatagcttagattaagacgctgagaagagtcaatagtgaatttatcaaaatcataggtctgagagactacctttttaacctccggcttaggttgggttatataactatatgtaaatgctgatgcaaatcctatcgcaagacaaagaacgcgagaaaactttttcaaatatattttagttaatttcatcttctgacctaaatttaatggtttgaaataccgaccgtgtgataaataaggggttaaatatgaataaacacctgaatcataattactggaaaaagcctgtttagtgtcatatgcgttatataaattcttaccagtataaagccaacgctcaacagtagggcttaattgagaatcaccatatttaacaacaccaacatgtaatttaggcagaggcatttttgagccagtaacaagagaatataaagtgccgacaaagggtaaagtaattctgtccagacggcgacaataaacaacgtgttcagctaatgcagaacgcgcctgtttatcaacaatagataaatcctgaacaagaaaaataatatcccatcccaatttacgagcatgaagaaaccaatcaataatcggctgtctttccttgtcattccatgaacgggtattaaaccaagtaccgcattcatcaagaacaagcaaaccgtttttattttcgtcgtaggaatcattaccacgaccaatagcaagcaaatcagaaatagaaggcttatccggtattctaagaacgcgaggcgttttagcgaacctcccgacttgcgggaggttttgaagccttaaatcaagattagttgctattttgcacccagctacaattttatcctgaatcttaccaacgctaacgagcgtctttccagagcctaatttgccagttacaaaataaacagccatatttatttatcccaatccaaataagaaacgattttttgtttaacgtcaaaaatgaaaatagcagcctttacagagagaataacataaaaacagggaagcgcattagacgggagaattaactgaacgccctgaacaaaatcagagggtaattgtgcgctaatatcagagagataacccacaagaattgagttaagcccaataataagagcaagaaacaatgaaatagcaatagctatcttaccgaagccctttttaaggaaagtaagcagatagccgaacaaagttaccagaaggaaaccgaggaaacgcaataataacggaatacccaaaagaactggcatgattaagactccttattacgcagtatgttagcaaacgtcgaaaatacatacataaaggtggcaacatataaaagaaacgcaaagacaccacggaataagtttattttgtcacaatcaatagaaaattcatatggtttaccagcgccaaagacataagggcgacattcaaccgactgaggcaaagaaggtaaatattgacggaaattattcattaaaggtgaattatcaccgtcaccgacttgagccatttgggaattagagccagcaaaatcaccagtagcaccattaccattagcaaggccggaaacgtcaccaatgaaaccatcgatagcagcaccgtaatcagtagcgacagaatcaagtttgcctttagcgtcagactgtagcgcgttttcatcggcattttcggtcatagcccccttattagcgtttgccattttttcataatcaaaatcaccggaaccggagccgccaccggaaccgccaccctcagagccgccaccctcagaaccgccaccctcagagccgccaccctcagagccgccaccagaaccaccaccagagccgccgccagcattgacaggaggttgaggcaggtcagacgattggccttgatattcacaaacgaatggatcctcattaaagccagaatggaaagcgcagtctctgaatttaccgttccagtaagcgtcatacatggcttttgatgatacaggagtgtactggtaataagttttaacggggtcagtgccttgagtaacagtgcccgtataaacagttaatgcaccctgcctatttcggaacctattattctgaaacatgaaagtattaagaggctgagactcctcaagagaaggattaggattagcggggttttgctcagtaccaggcggataagtgccgtcgagagggttgatataagtatagcccggaataggtgtatcaccgtactcaggaggtttagtaccgccaccctcagaaccgccaccctcagaaccgccaccctcagagccaccaccctcattttcagggatagcaagcccaataggaacccatgtaccgtaacactgagtttcgtcaccagtacaaaccacaacgcctgtagcattccacagacagccctcatagttagcgtaacgatctaaagttttgtcgtctttccagacgttagtaaatgaattttctgtatgaggttttgctaaacaactttcaacagtttcagcggagtgagaatagaaaggaacaactaaaggaattgcgaataataattttttcacgttgaaaatctccaaaaaaaaaggctccaaaaggagcctttaattgtatcggtttatcagcttgctttcgaggtgaatttcttaaacagcttgataccgatagttgcgccgacaatgacaacaaccatcgcccacgcataaccgatatattcggtcgctgaggcttgcagggagtcaaaggccgcttttgcgggatcgtcaccctcagcagcgaaagacagcatcggaacgagggtagcaacggctacggaggctttgaggactaaagactttttcatgaggaagtttccattaaacgggtaaaatacgtaatgccactacgaaggcaccaacctaaaacgaaagaggcgaaagaatacactaaaacactcatctttgacccccagcgattataccaagcgcgaaacaaagtacaacggagatttgtatcatcgcctgataaattgtgtcgaaatccgcgacctgctccatgttacttagccggaacgaggcgcagacggtcaatcataagagaaccgaactgaccaactttgaacgaggacagatgcacggtgtacagaccaggcgcatacgctggctgaccttcgtcgagagtaatcttgacaagcaccggatattcattacccaaatcaacgtaacaaagctgctcattcagtgaataaggcttgccctgacgagaaacaccagaacgggtagtaaattgcgcttgagacggtttaatttcaactttaatcattttgaattaccttatgcgattttaagaactggctcattataccagtcaggacgttgggaggaaaaatctacgttaataaaacgaactaacggaacaacattattacaggtggaaagattcatcaattgagatttaggaataccacactcaactaatgcagatacataacgccaaaaggaattacgaggcatggtaagagcaacactatcataaccctcattaaccagacgacgatagaaaccaaaatagcgagaggcttttgcaaaggaagttttgccagagggggtaattgtaaaatgtttagactggatagcgtccaatactgcggaatcgtcataaatattcattgaatccccctcaaatgctttaaacagttcagaaaacgagaatgaccataaatcaaaaatcaggtctttaccctgtctattatagtcagaagcaaagcgaattgcatcaaaaagattaagaggaagcccgaaagacttcaaatatcgcgtttcaattcgagcctcaaagcgaaccagaccggaagcaaattccaacaggtcaggattagacagtacctttaattgctccttttgataagaggtcatttttgcggatggcttagagcttaattgctgaatctggtgctgtagttcaacatgttttaaatatgcaactaaagtacggtgtctggaagtttcattccatgtaacagttgattcccaattctgcgaacgagtagatttagtttgaccattagatacatttcgcaaatggtcaataacctgtttagctatattttcatttggggcgcgagctgaaaaggtggcatcaattctactaatggtagtagcgttaacatccaataaatcgtacaagcaaggcagagagttagcaaaattaagcaataaagcctcagagcataaagctaaatcggttgtaccaaaaacattatgaccctgtaatacttttgctggtgaagccttaatttcaacgcagggataaaaatttttagaaccctcatatattttaaatgcaatgccggagtaatgagtaggcaaagattcaaacgggtgagaaaggccggagacagtcaaatcaccgtcaatatgatattcaaccgttctagctgataaattcatgccggagagggtagctatttttgagaggtctacaaaggctatcaggtcattacctgaaagtctggagcaaacaagagaatcgatgaacggtaatcgtaaaactagcatgtcaatcatatgtaccccggttgataatcagaaaagccccaaaaacaggatgattgtataagcaaatatttaaattgtaaacgttaatgttttgttaaatttcgcgttaaatatttgttaaatcagcttattttttaaccagtaagcagaaaatgacaaaaatccttataaatcaaaagaataggccgagttagttgtgagtgttgttccagtttggaacaagagtccactatt